(19) United States
(12) Patent Application Publication
(10) Pub. No.: US 2025/0185800 A1
(43) Pub. Date: Jun. 12, 2025

(54) MEDICAL TOOLS FOR INTERACTING WITH LUMENS

(71) Applicant: Jacek KRZYZANOWSKI, Etobicoke (CA)

(72) Inventor: Jacek KRZYZANOWSKI, Etobicoke (CA)

(21) Appl. No.: 18/842,303

(22) PCT Filed: Jul. 28, 2022
(86) PCT No.: PCT/CA2022/051159

§ 371 Date: Aug. 28, 2024

Related U.S. Application Data
(60) Provisional application No. 63/316,494, filed on Mar. 4, 2022.

(51) Int. Cl. A46B 15/00; A46B 3/16; A46B 9/02; A46D 3/00; A61B 90/70

(52) U.S. Cl. CPC A46B 15/0055; A46B 3/16; A46B 9/026; A46D 3/005; A61B 90/70; A46B 2200/3013

(57) ABSTRACT

Medical lumen interacting tools and associated manufacturing methods are provided. A medical lumen interacting tool comprises an elongated core having a longitudinal axis, a body attached to the elongated core for interacting with the lumen, and one or more anchors interlocking the body with the elongated core along the longitudinal axis of the elongated core to hinder relative movement between the elongated core and the body along the longitudinal axis.

### CROSS REFERENCE TO RELATED APPLICATION(S) AND CLAIM OF PRIORITY

The present application claims priority to U.S. provisional patent application No. 63/316,494 filed on Mar. 4, 2022, the entire contents of which are hereby incorporated herein by reference.

### TECHNICAL FIELD

The disclosure relates generally to medical tools, and more particularly to medical tools for interacting with lumens of medical devices and/or for interacting with anatomical or artificial lumens.

### BACKGROUND

Some medical tools include an elongated core with one or more operational bodies attached thereto for interacting with lumens. Such medical tools may be configured to be inserted into and moved along a lumen of a medical device or an anatomical lumen. During use, the operational body(ies) should not unintentionally become dislodged from the elongated core.

### SUMMARY

In one aspect, the disclosure describes a lumen cleaning tool for cleaning a lumen of a medical device. The lumen cleaning tool comprises:
an elongated core having a longitudinal axis, the elongated core being defined by wires twisted together;
a wiper attached to the elongated core; and
an anchor interlocking the wiper with the elongated core along the longitudinal axis of the elongated core to hinder relative movement between the elongated core and the wiper along the longitudinal axis.

The elongated core may include a tightly twisted section and a loosely twisted section having different axial positions along the elongated core. The loosely twisted section may define a gap between the wires twisted together. The wiper may be overmolded onto the loosely twisted section of the elongated core. The anchor may include a beam extending into the gap. The beam and the wiper may have a monolithic construction.

The gap may define a through passage for the beam to extend transversely through the elongated core.

The anchor may include a protuberance attached to the elongated core.

The wiper may at least partially encapsulate the protuberance.

The protuberance and the wiper may be made from different materials.

The protuberance may include a bristle retained between the wires twisted together.

The protuberance may be disc-shaped. The protuberance may occupy space inside a radially-extending fin of the wiper.

The wiper may include an overmolded body overmolded onto the elongated core and onto the protuberance. The protuberance may protrude out of the overmolded body.

The anchor may include a helical arrangement of bristles extending radially outwardly from the elongated core. The wiper may at least partially encapsulate the helical arrangement of bristles.

The lumen cleaning tool may comprise a plurality of anchors interlocking the wiper with the elongated core along the longitudinal axis of the elongated core. The plurality of anchors may be axially spaced apart along the longitudinal axis of the elongated core.

The plurality of anchors may include a first anchor having a first radial dimension from the longitudinal axis of the elongated core, and a second anchor having a second radial dimension from the longitudinal axis of the elongated core. The second radial dimension may be greater than the first radial dimension.

The anchor may be crimped onto the elongated core. The anchor may be welded to the elongated core. The anchor may be overmolded onto the elongated core.

The anchor may include a tube having an inner bore. The elongated core may include a first portion inside of the inner bore of the tube. The elongated core may include a second portion outside of the inner bore of the tube. The wiper may be overmolded onto the second portion of the elongated core and onto the tube.

A longitudinal length of the tube may be greater than a longitudinal length of the first portion of the elongated core inside of the tube.

The anchor may be made of polypropylene. The wiper may be made of a thermoplastic elastomer.

In another aspect, the disclosure describes a medical lumen interacting tool for interacting with a lumen. The medical lumen interacting tool comprises:
an elongated core having a longitudinal axis;
a body for interacting with the lumen, the body being attached to the elongated core; and
one or more anchors interlocking the body with the elongated core along the longitudinal axis of the elongated core to hinder relative movement between the elongated core and the body along the longitudinal axis, the one or more anchors including one or both of following:
a beam having a monolithic construction with the body and extending in a passage formed through the elongated core; and
a protuberance attached to the elongated core and being at least partially encapsulated by the body.

The one or more anchors may include the beam.

The elongated core may include wires twisted together. The passage may be defined by a gap between the wires.

The elongated core may include a tightly twisted section and a loosely twisted section. The passage may be located in the loosely twisted section.

The body may be a first body. The medical lumen interacting tool may include a second body for interacting with the lumen. The second body may be attached to the elongated core and axially located in the tightly twisted section of the elongated core.

The elongated core may include a helical coil. The passage may be defined by a gap defined between two turns of a wire of the helical coil.

The helical coil may include a first section having a first pitch and a second section having a second pitch larger than the first pitch. The first section may be axially adjacent the second section along the helical coil. The body may be attached to the helical coil and may be axially located in the second section of the of the helical coil.

The first section of the helical coil and the second section of the helical coil may have a substantially same outer diameter.

The wire may be sole wire of the elongated core.

The wire may have a polygonal cross-sectional profile.

The wire may have a circular cross-sectional profile.

The one or more anchors may include the protuberance.

The protuberance may include a bristle.

The protuberance may have a helical shape.

The protuberance may include a helical arrangement of bristles extending radially outwardly from the elongated core.

The protuberance may include a disc.

The protuberance may be ring-shaped.

The protuberance may include a tube having an inner bore. The elongated core may include a first portion inside of the inner bore of the tube. The elongated core may include a second portion outside of the inner bore of the tube. The body may be overmolded onto the second portion of the elongated core and onto the tube.

A longitudinal length of the tube may be greater than a longitudinal length of the first portion of the elongated core inside of the tube.

The anchor may be made of polypropylene.

The body may be made of a thermoplastic elastomer.

The elongated core may include a helical coil.

The helical coil may include a first section having a first outer diameter and a second section having a second outer diameter larger than the first outer diameter. The protuberance may include the second section.

The helical coil may include a third section having a third outer diameter smaller than the second outer diameter of the second section. The second section may be located axially between the first section and the third section.

The second section may have an axial span smaller than an axial span of the first section.

The elongated core may have a sole wire defining the helical coil.

The body may be overmolded onto the elongated core.

The body may be a wiper.

The body may be a lumen stabilizer.

In a further aspect, the disclosure describes a method of manufacturing a medical lumen interacting tool for interacting with a lumen. The method comprises:
receiving an elongated core having a longitudinal axis; and
interlocking a lumen interacting body with the elongated core to hinder relative movement between the elongated core and the lumen interacting body along the longitudinal axis by performing one or both of following:
overmolding the lumen interacting body onto the elongated core using overmolding material, the overmolding material defining a beam extending in a passage formed through the elongated core; and
at least partially encapsulating a protuberance attached to the elongated core with the lumen interacting body.

Interlocking the lumen interacting body with the elongated core may include overmolding the lumen interacting body onto the elongated core using overmolding material. The overmolding material may define the beam extending through the passage formed through the elongated core.

The elongated core may include a pair of wires twisted together. The passage may be defined by a gap between the pair of wires.

The elongated core may include a tightly twisted section and a loosely twisted section. The passage may be located in the loosely twisted section.

Interlocking the lumen interacting body with the elongated core may include at least partially encapsulating the protuberance attached to the elongated core with the lumen interacting body.

At least partially encapsulating the protuberance attached to the elongated core with the lumen interacting body may include ovemolding the lumen interacting body onto the elongated core and onto the protuberance.

The protuberance may include a bristle.

The method may include welding, crimping, or overmolding the protuberance to the elongated core.

The elongated core may include a helical coil.

The protuberance may include a tube having an inner bore. The method may include inserting the elongated core into the inner bore of the tube before at least partially encapsulating the tube with the lumen interacting body.

The method may include overmolding the lumen interacting body onto the elongated core and onto part of the tube.

The method may include injecting overmolding material between the elongated core and the inner bore of the tube.

Further details of these and other aspects of the subject matter of this application will be apparent from the detailed description included below and the drawings.

### DESCRIPTION OF THE DRAWINGS

Reference is now made to the accompanying drawings, in which:

FIG. 1 is a perspective view of an exemplary lumen cleaning tool for cleaning lumens of medical devices;

FIG. 2 is a side elevation view of the tool of FIG. 1;

FIG. 3 is a side elevation view of the tool of FIG. 1 disposed inside a lumen of a medical device and being propelled inside the lumen;

FIG. 4 is a side elevation view of another exemplary lumen cleaning tool disposed inside a lumen of a medical device and being propelled inside the lumen;

FIG. 5 is a side elevation view of a scrubber of the lumen cleaning tool of FIG. 4;

FIG. 5A is a magnified view of bristles of the scrubber of FIG. 5 in region A in FIG. 5;

FIG. 6 is a side elevation view of another exemplary lumen cleaning tool;

FIG. 7 is a flow diagram of a method for cleaning a lumen of a medical device;

FIG. 8 is a flow diagram of a method of propelling a lumen cleaning tool along a lumen of a medical device using a pressure differential;

FIG. 9 is a side view of the lumen cleaning tool of FIG. 1 disposed inside a lumen of a medical device and propelled along the lumen using a pressure differential;

FIG. 10 is a perspective view of another exemplary lumen cleaning tool for cleaning lumens of medical devices;

FIG. 11 is a side view of part of the lumen cleaning tool of FIG. 10 disposed inside and being propelled along a curved lumen of a medical device;

FIG. 12 is a schematic illustration of an exemplary wiper of the lumen cleaning tool of FIG. 10 being released from a mold where the wiper is illustrated as being transparent to show an elongated core disposed inside the wiper;

FIG. 13 is a side view of part of the elongated core of the lumen cleaning tool of FIG. 10 shown in isolation;

FIG. 14 is a schematic perspective view of part of the elongated core of FIG. 13 during manufacturing of the elongated core;

FIG. 15A is a cross-sectional view of the lumen cleaning tool of FIG. 12 taken along line 15-15 of FIG. 12;

FIGS. 15B and 15C are cross-sectional views of other exemplary lumen cleaning tools of the type shown in FIG. 12 taken along line 15-15 of FIG. 12;

FIG. 16 is a side view of another exemplary lumen cleaning tool for cleaning lumens of medical devices where a wiper of the lumen cleaning tool is illustrated as being transparent to show an elongated core with bristles attached thereto interlocking the wiper with the elongated core;

FIG. 17 is a side view of an elongated core and the bristles of the lumen cleaning tool of FIG. 16;

FIG. 18 is a side view of another exemplary lumen cleaning tool for cleaning lumens of medical devices where a wiper of the lumen cleaning tool is illustrated as being transparent to show an elongated core with ring-shaped anchors attached thereto interlocking the wiper with the elongated core;

FIG. 19 is a side view of the elongated core and the ring-shaped anchors of the lumen cleaning tool of FIG. 18;

FIG. 19A is a magnified side view of a ring-shaped anchor crimped onto the elongated core of FIG. 19;

FIG. 19B is a magnified side view of a ring-shaped anchor welded to the elongated core of FIG. 19;

FIG. 20 is a side view of part of another exemplary lumen cleaning tool for cleaning lumens of medical devices where a wiper of the lumen cleaning tool is illustrated as being transparent to show an elongated core with anchors welded thereto interlocking the wiper with the elongated core;

FIG. 21 is a side view of the elongated core and the anchors of the lumen cleaning tool of FIG. 20;

FIG. 22 is a side view of part of another exemplary lumen cleaning tool for cleaning lumens of medical devices where a wiper of the lumen cleaning tool is illustrated as being transparent to show an elongated core with anchors welded and crimped thereto interlocking the wiper with the elongated core;

FIG. 23 is a side view of the elongated core and the anchors of the lumen cleaning tool of FIG. 22;

FIG. 24 is a side view of part of another exemplary lumen cleaning tool for cleaning lumens of medical devices where a wiper of the lumen cleaning tool is illustrated as being transparent to show an elongated core with anchors attached thereto interlocking the wiper with the elongated core;

FIG. 25 is a side view of the elongated core and anchors of the lumen cleaning tool of FIG. 24;

FIG. 26 is a perspective view of an exemplary lumen stabilizing tool for stabilizing a lumen;

FIG. 27 is a side view of part of the lumen stabilizing tool of FIG. 26 where a stabilizer of the lumen stabilizing tool is illustrated as being transparent to show an elongated core with bristles attached thereto interlocking the stabilizer with the elongated core;

FIG. 28 is a side view of the elongated core and bristles of the lumen stabilizing tool of FIG. 26;

FIG. 29 is a side view of part of another exemplary lumen stabilizing tool where a stabilizer of the lumen stabilizing tool is illustrated as being transparent to show an elongated core including an exemplary helical coil;

FIG. 30 is a side view of the elongated core of the lumen stabilizing tool of FIG. 29 shown in isolation;

FIG. 31 is an enlarged side view of part of the helical coil of FIG. 30 and material from the stabilizer;

FIG. 32 is a side view of part of another exemplary lumen stabilizing tool where a stabilizer of the lumen stabilizing tool is illustrated as being transparent to show an elongated core including another exemplary helical coil;

FIG. 33 is a side view of the elongated core of the lumen stabilizing tool of FIG. 32 shown in isolation;

FIG. 34 is a side view of part of another exemplary lumen stabilizing tool where a stabilizer of the lumen stabilizing tool is illustrated as being transparent to show an elongated core including another exemplary helical coil;

FIG. 35 is a side view of the elongated core of the lumen stabilizing tool of FIG. 34 shown in isolation;

FIGS. 36A and 36B illustrate exemplary two-shot overmolding method for manufacturing a lumen cleaning tool;

FIG. 37A is a perspective view of an exemplary anchor attached to an elongated core;

FIG. 37B is a side view of an exemplary lumen interacting tool including a lumen interacting body partially encapsulating the anchor of FIG. 37A;

FIG. 37C is a cross-sectional view of the lumen interacting tool of FIG. 37B taken along line 37C-37C in FIG. 37B;

FIG. 38 is a side view of another exemplary lumen cleaning tool for cleaning lumens of medical devices including a tube interlocking the wiper with the elongated core;

FIG. 39 is a schematic illustration of part of the lumen cleaning tool inside region 39 of FIG. 38 showing a wiper being overmolded onto a portion of an elongated core and a portion of the tube;

FIG. 40 is an enlarged view of part of lumen cleaning tool in region 40 of FIG. 38; and

FIG. 41 is a flow diagram of a method of manufacturing a lumen interacting tool for interacting with a lumen.

### DETAILED DESCRIPTION

The following disclosure describes medical tools and associated methods for interacting with (e.g., cleaning, contacting, stabilizing) lumens of reusable medical (e.g., surgical) devices such as endoscopes, and/or anatomical lumens such as gastrointestinal tracts or blood vessels of a cardiovascular system of a human or animal for example. In some embodiments, the medical tools described herein may include one or more operational lumen interacting bodies (e.g., lumen wipers, lumen stabilizers such as implantable stents, stent-like devices or other implants) that are attached to an elongated core and interlocked with the elongated core via one or more anchors. The anchor(s) may enhance the retention of the lumen interacting body(ies) onto the elongated core for overcoming friction force(s) between the lumen interacting body(ies) and the lumens when the tools are inserted into and propelled inside the lumens.

In some embodiments, the interlocking may be provided by mechanical and/or adhesive locking between a lumen interacting body and an anchor. In other words, movement of the lumen interacting body along the elongated core may be constrained by the anchor. For example, the interlocking may be provided by way of the lumen interacting body and the anchor fitting into each other so that movement of the lumen interacting body and the anchor may be coordinated (e.g., synchronized). For example, the interlocking may be provided by way of the lumen interacting body and the anchor being interweaved or interlaced with one another. In some embodiments, the interlocking may be provided by way of a mutual interconnection of one or more parts between the lumen interacting body and the anchor. In some embodiments, the interlocking may be provided by suitable (e.g., melt) adhesion between the lumen interacting body and the anchor.

In some embodiments, the anchor(s) may prevent migration of an implantable lumen stabilizing body inside of a lumen. The enhanced retention of the lumen interacting body(ies) onto the elongated core may reduce the potential for the lumen interacting body(ies) unintentionally becoming dislodged from the elongated core during use. In some embodiments, the enhanced retention of the lumen interacting body(ies) onto the elongated core may reduce the potential of damaging medical devices that are being cleaned, and promote patient safety when the medical tool is used inside an anatomical lumen.

In some embodiments, the types and configurations of anchors used to interlock the lumen interacting body(ies) with the elongated core may also allow flexibility of the medical tool to permit the medical tool to be inserted into and be propelled along a curved lumen for example. In the various tools shown in the accompanying drawings, the elongated cores may be longer than the portions shown, and the tools may include additional elements attached to the elongated cores.

Aspects of the present disclosure including various means of interlocking lumen interacting body(ies) with elongated cores may be applied to various medical tools including lumen cleaning tools such as those described in U.S. Patent Publication No. 2021/0244266 (Title: TOOLS AND METHODS FOR CLEANING LUMENS OF MEDICAL DEVICES), which is incorporated herein by reference.

Embodiments of medical tools described herein include different types of lumen interacting bodies as non-limiting examples. Anchoring and manufacturing methods described herein in relation to one type of lumen interacting body (e.g., lumen wiper) may be suitable for other types of lumen interacting bodies (e.g., lumen stabilizer).

The term “attached” may include both direct attachment (in which two elements contact each other) and indirect attachment (in which at least one additional element is located between the two elements).

The term “substantially” as used herein may be applied to modify any quantitative representation which could permissibly vary without resulting in a change in the basic function to which it is related.

Aspects of various embodiments are described through reference to the drawings.

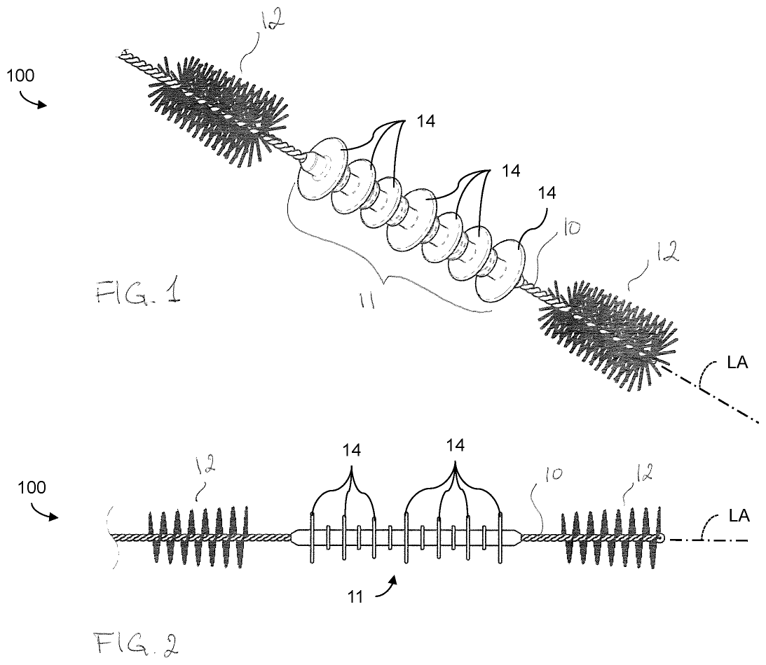

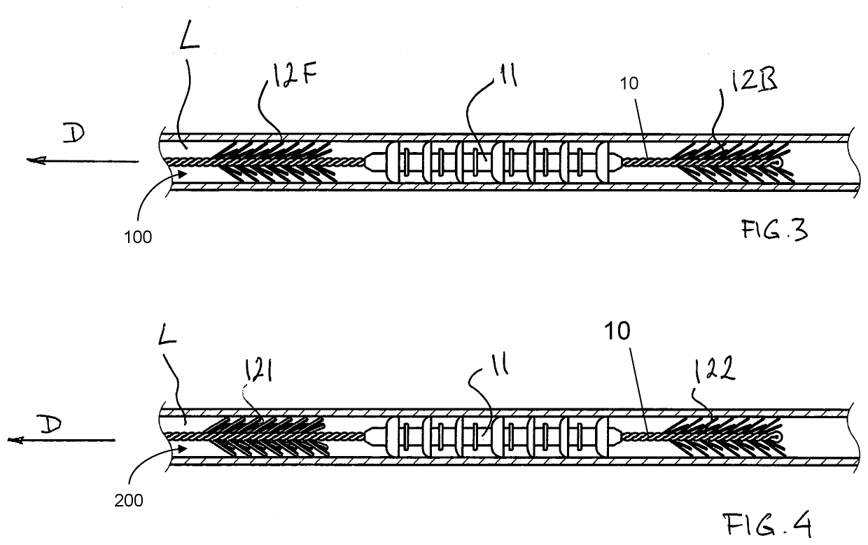

FIGS. 1 and 2 are respective perspective and side elevation views of an exemplary lumen cleaning tool 100 (referred hereinafter as “tool 100”). Tool 100 may comprise elongated core 10 (referred hereinafter as “core 10”), one or more wipers 11 and one or more scrubbers 12. Core 10 may have a longitudinal axis LA and may have an axial length that is greater than that of the portion of core 10 shown in FIG. 1. Wiper 11 and scrubbers 12 may be attached to core 10. Wiper 11 may be located axially between two scrubbers 12 along the length of core 10. Core 10 may include a wire, twisted wires, a tube, a rod, a catheter, or a combination thereof. Core 10 may be of a unitary construction or may include multiple elements (e.g., axial sections) assembled together. Core 10 may be flexible, semi-rigid or substantially rigid along its entire axial length. Core 10 may have a non-uniform flexibility that varies along its length. As an example, core 10 may include a flexible section followed by a rigid section. An exemplary core 10 shown in FIG. 1 includes (e.g., a pair of) twisted (e.g., metallic, stainless steel) wires.

Core 10 may be of any length and thickness (e.g., diameter) that suit the size and configuration of the lumen to be cleaned, and that accommodate the desired number and configuration of wipers 11 and scrubbers 12 included in tool 100. In some non-limiting embodiments, core 10 may have a thickness (e.g., diameter) that is between 0.3 mm and 2.0 mm and may have an axial length that is between 50 mm and 2500 mm.

Wipers 11 and scrubbers 12 may be attached to and positioned along core 10 of cleaning tool 100. Wipers 11 and scrubbers 12 may be sized to allow contact (i.e., frictional engagement) with the lumen's wall(s) for mechanically removing the contaminants (e.g., debris, solids, liquids) inside of the lumen. In other words, the outer diameters of wipers 11 and scrubbers 12 may be larger than the diameter of the lumen to provide an interference fit within the lumen as shown in FIGS. 3, 4 and 9.

Wiper 11 may include one or more (e.g., a set of) flexible disc-shaped squeegees 14 as shown in FIG. 1 that may be made of a shape-conforming thermoplastic. For example, squeegees 14 may be made of a suitable thermoplastic elastomer (TPE), thermoplastic rubber (TPR), thermoplastic polyurethane (TPU) and/or thermoplastic vulcanizate (TPV). Squeegees 14 may be flexible radially-extending fins that are axially distributed along core 10. Alternatively or in addition, wiper 11 may include one or more O-rings made of silicon or any other suitable material, one or more sponges made of a moisture-expanding material, and/or one or more inflatable balloons configured to maintains close contact with the wall(s) defining the lumen. Wiper 11 may be continuous along its length along longitudinal axis LA of core 10. Alternatively, wiper 11 may include a plurality of axial sections separated by axial gaps exposing core 10. In various embodiments, wiper 11 may be attached to core 10 by suitable means (e.g., adhesive), or may be overmolded onto core 10 using injection molding as explained below for example.

The dimensions of wiper 11 may be of any length and thickness that suit the size of the lumen to be cleaned. In some non-limiting embodiments, wiper 11 may have a thickness (e.g., diameter) that is between 1 and 15 mm and a length that is between 20 mm and 100 mm.

Scrubbers 12 may include twist-in wire (e.g., bristle) brushes attached to core 10 as shown in FIG. 1. Scrubbers 12 may include twisted wire brushes including bristles that are held in place by, and which extend radially from, twisted wire core 10. The bristles may also be circumferentially distributed around core 10. To form the twisted wire brush, the bristles may be inserted between parallel wires and then the wires may be twisted together to press (clamp) and retain the bristles therebetween. Other suitable methods of attaching bristles to core 10 of different types may be used. Depending on the intended application, the density of the bristles and the surface area covered by the bristles may be varied by adjusting the number of bristles and/or by angling the bristles at desired angles from longitudinal axis LA.

The bristles may be made of suitable materials (e.g., polyester, nylon, polypropylene) having suitable physical dimensions, flexibility, and other characteristics according to the intended application. In general, the dimensions of the scrubbers 12 can be of any length and thickness that suit the size of the lumen to be cleaned. In some non-limiting embodiments, the scrubbers 12 may have a thickness (e.g., diameter) that is between 1 mm and 15 mm and an axial length that is between 5 mm and 100 mm. Scrubbers 12 may be continuous along their length along longitudinal axis LA of core 10. Alternatively, one or more both scrubbers 12 may include a plurality of axial sections separated by axial gaps exposing the core 10.

Alternatively or in addition, scrubbers 12 may include one or more abrasive sponges, molded brushes, abrasive fibers, or any other elements containing a scrubbing media. Scrubbers 12 may be attached to and positioned along core 10 of tool 100 and may be sized to allow contact between the scrubbers 12 and the wall(s) defining the lumen.

Tool 100 may also incorporate other features (not shown), such as size limiters preventing tool 100 entry into (e.g., undersized) lumens of predetermined sizes or shapes, and/or leaders allowing easy entry of tool 100 into lumens.

FIG. 3 is a side view of tool 100 disposed inside lumen L of a medical device and being propelled inside lumen L in direction D. FIG. 3 shows an axial cross-sectional view of lumen L to expose tool 100 disposed therein. Lumen L may be a channel of a flexible or rigid endoscope for example. During operation, tool 100 may be propelled inside lumen L either manually, semi-automatically or automatically so that wiper 11 and scrubbers 12 may cooperatively expel the contaminants out of lumen L. For example, tool 100 may be manually pushed or pulled through lumen L by manually pushing or pulling on core 10. Alternatively or in addition, tool 100 may be propelled through lumen L by way of an actuator drivingly coupled to tool 100, and/or by way of a pressure differential (e.g., positive or negative pressure) axially across wiper 11 as explained further below for example.

Tool 100 may include wiper 11 that is disposed axially between front scrubber 12F and back scrubber 12B in relation to direction D of movement relative to lumen L. As tool 100 is moved axially inside lumen L in direction D by pushing or pulling tool 100 along the lumen L, front scrubber 12F is disposed ahead of wiper 11 and scrubs and loosens the contaminants from the wall(s) defining lumen L. The wiper 11 then wipes (e.g., squeegees) the contaminants and moves them forward in the direction D of the axial movement of tool 100 and eventually expels them out of lumen L. However, the frictional engagement of wiper 11 with the wall(s) defining lumen L may, in some situations, cause a relatively thin biofilm to be left behind on the wall(s) of lumen L. Such biofilm could be difficult to remove in one or more subsequent decontamination steps. Furthermore, the wall(s) defining lumen L may not be perfectly smooth. For example, lumen L may have surface imperfections such as scratches and voids. Wiper 11 may push some contaminants into these surface imperfections, making them even more difficult to remove in subsequent decontamination steps such as rinsing/flushing with detergent-based solutions.

In some situations, the use of back scrubber 12B being propelled behind wiper 11 along direction D may facilitate subsequent decontamination steps by scrubbing and loosening the residual biofilm or other contaminants that may be left behind wiper 11. Such scrubbing and loosening of the contaminants behind wiper 11 may enhance the efficiency of the subsequent rinsing/flushing steps that may be involved in cleaning or decontaminating lumen L. Accordingly, the combination of scrubbers 12F, 12B and wiper 11 disposed axially therebetween may cause a series of cooperating steps to be carried out in a sequence that enhances cleaning, and that may also facilitate subsequent decontamination of lumen L in some situations.

In some embodiments, scrubbers 12F, 12B may have substantially identical constructions. For example, scrubbers 12F, 12B may have a same density of bristles, and may also have bristles of the same cross-sectional thickness (e.g., diameter) and length. Alternatively, scrubbers 12F, 12B may have different constructions as explained below.

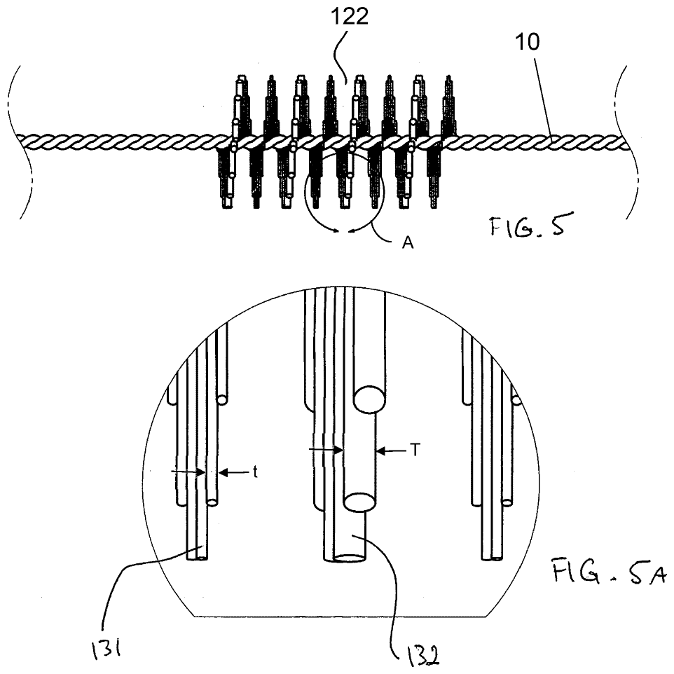

FIG. 4 is a side view of another exemplary lumen cleaning tool 200 (referred hereinafter as “tool 200”) disposed inside lumen L of a medical device and being propelled inside lumen L along direction D. FIG. 4 shows an axial cross-sectional view of lumen L to expose tool 200 disposed therein. Tool 200 may include core 10, scrubbers 121, 122 and wiper 11 disposed axially between scrubbers 121, 122. Scrubbers 121, 122 may be bristle brushes. In contrast with tool 100, scrubbers 121 and 122 of tool 200 may have different constructions from each other. For example, the bristles in front scrubber 121 and back scrubber 122 may have different cross-sectional thicknesses (e.g., diameters). For example, back scrubber 122 may have bristles of smaller thickness than those of front scrubber 121. For example, back scrubber 122 may be a relatively fine bristle brush and front scrubber 121 may include a relatively coarse bristle brush.

Front scrubber 121 may have (i.e., coarser) bristles of a larger thickness that, during operation, more aggressively scrub and loosen the contaminants from the wall(s) defining lumen L ahead of wiper 11. On the other hand, back scrubber 122 may have (i.e., finer) bristles of a smaller thickness that may interact more desirably with the thin scratches and small voids in the lumen's wall. Accordingly, the bristles of smaller thickness of back scrubber 122 may be better adapted to scrub and release contaminants from relatively small surface imperfections in the lumen's wall(s) and may facilitate subsequent cleaning or decontamination steps.

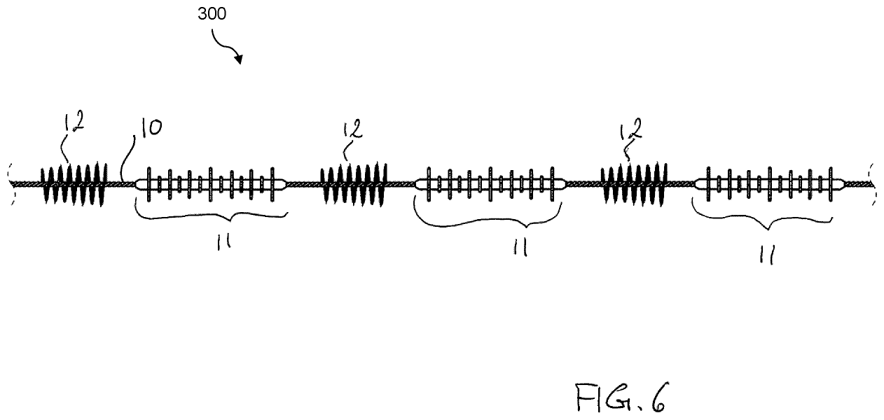

FIG. 5 is a side view of an exemplary back scrubber 122 of tool 200 shown of FIG. 4. FIG. 5A is a magnified view of bristles 131, 132 of back scrubber 122 in region A of FIG. 5. In some embodiments, back scrubber 122 may include a combination of bristles 131 of smaller thickness t and bristles 132 of larger thickness T that may cooperate together to provide desired scrubbing action on the wall(s) defining lumen L. The arrangement of bristles 131, 132 in back scrubber 122 can be such that sections of thinner bristles 131 and thicker bristles 132 are alternatively arranged along core 10. Alternatively, thinner bristles 131 and thicker bristles 132 may be randomly arranged or otherwise mixed within back scrubber 122. Back scrubber 122 may contain bristles of two or more (e.g., three, four or five) different thicknesses. The cross-sectional profile(s) of bristles 131, 132 may be circular, oval, rectangular or any other suitable shape. Bristles 131, 132 may be solid or hollow.

During operation, thicker bristles 132 of back scrubber 122 may more aggressively scrub and loosen the biofilm that may be left behind wiper 11, and thinner bristles 131 may scrub and help release or loosen contaminants lodged in the surface imperfections formed in the lumen's wall(s) to facilitate subsequent cleaning or decontamination of lumen L.

In various embodiments of scrubbers described herein that include bristle brushes, suitable bristle thicknesses (e.g., diameters) and materials may be selected based on the specific application and anticipated cleaning needs. For example, the bristles may be made from nylon or other suitable material(s). The bristles of the scrubbers described herein may be of uniform or non-uniform thicknesses. Examples of suitable bristle thicknesses (e.g., diameters) in some applications may be about 0.001″ (0.03 mm), about 0.002″ (0.05 mm), about 0.003″ (0.08 mm), about 0.004″ (0.10 mm), about 0.005″ (0.13 mm), about 0.006″ (0.15 mm), about 0.007″ (0.18 mm), about 0.008″ (0.20 mm), about 0.009″ (0.23 mm) or about 0.010″ (0.25 mm) for example.

FIG. 6 is a side view of another exemplary lumen cleaning tool 300 (referred hereinafter as “tool 300”). Tool 300 may have components previously described above in relation to tool 100. Like elements are identified using like reference numerals. In some embodiments, tool 300 may include a plurality of scrubbers 12 and a plurality of wipers 11. Scrubbers 12 and wipers 11 may be alternatingly disposed one after the other along core 10. Accordingly, as tool 300 is propelled through lumen L, the arrangement of scrubbers 12 and wipers 11 may perform alternating scrubbing and wiping operations on the wall(s) defining lumen L.

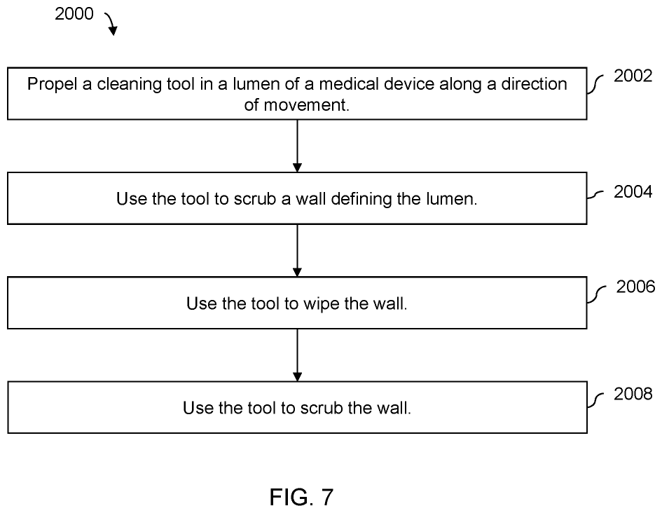

FIG. 7 is a flow diagram of method 2000 for cleaning lumen L of a medical device. Method 2000 may be performed with lumen cleaning tools described herein or with other lumen cleaning tools. Aspects or steps associated with lumen cleaning tools described herein may be incorporated into method 2000. Method 2000 may include:
propelling tool 100, 200 or 300 in lumen L along direction D (block 1002); and
while propelling tool 100, 200 or 300 in lumen L along direction D, using tool 100, 200 or 300 to, in a single pass of tool 100, 200 or 300 in lumen L,
scrub a wall defining lumen L (block 1004);
after scrubbing the wall, wipe the wall (block 1006); and
after wiping the wall, scrub the wall (block 1008).

In various embodiments of method 2000, tool 100, 200 or 300 may be passed a single time or multiple times through lumen L to achieve the desired cleaning level. However, the configurations of tool 100, 200 or 300 may permit the actions of initial scrubbing, wiping and subsequent scrubbing to be performed in a single pass of tool 100, 200 or 300 through lumen L. The scrubbing-wiping-scrubbing sequence of operations carried out in a single pass may be achieved by way of wiper 11 being axially disposed between two scrubbers 12. Propelling of tool 100, 200 or 300 may be performed manually and/or may be performed using a pressure differential inside of lumen L as explained further below.

After having cleaned lumen L using tool 100, 200 or 300, lumen L may be decontaminated using one or more subsequent steps such as rinsing/flushing with a detergent solution.

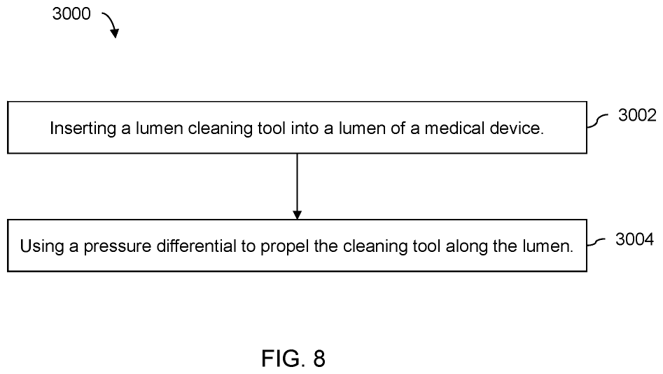

FIG. 8 is a flow diagram of method 3000 of propelling a lumen cleaning tool along lumen L of a medical device using a pressure differential. Method 3000 may be performed with lumen cleaning tools described herein or with other lumen cleaning tools. Aspects or steps associated with lumen cleaning tools described herein may be incorporated into method 3000. Aspects of method 3000 may be incorporated into method 2000. Method 3000 may be used in conjunction with lumen cleaning tools that include one or more scrubbers 12 and/or one or more wipers 11. Method 3000 may include:
inserting tool 100, 200 or 300 into lumen L (block 3002); and
when tool 100, 200 or 300 is disposed inside lumen L, using a pressure differential inside lumen L to propel tool 100, 200 or 300 along lumen L (block 3003).

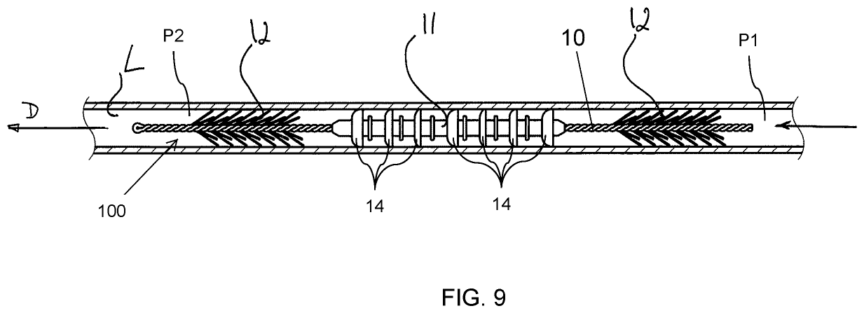

FIG. 9 is a side view of tool 100 disposed inside lumen L of a medical device and propelled inside lumen L using a pressure differential in accordance with method 3000. As wiper 11 maintains a close contact with the wall(s) of lumen L, wiper 11 may act as a piston such that when a positive or negative pressure is applied at one end of the lumen L, tool 100 may be propelled along lumen L. In other words, disc-shaped squeegees 14 may provide a substantially complete circumferential seal inside lumen L so that a sufficient pressure differential (e.g., ΔP=P1−P2) axially across wiper 11 may urge and propel tool 100 along lumen L.

In reference to FIG. 9, a sufficient difference between pressure P1 and pressure P2 (i.e., P1>P2) may cause movement of tool 100 along lumen L. For example, the application of a positive pressure P1 behind wiper 11 (e.g., using a source of pressurized air or water) may be used to push tool 100 along lumen L in direction D. Instead or in addition, the application of a negative pressure P2 in front of wiper 11 (e.g., using a vacuum pump) may be used to pull tool 100 along lumen L in direction D.

The pressure differential may be applied by way of a pressurized fluid (e.g., gas and/or liquid) including air and/or water. For example, water pressurized to about 40 psi (276 kPa) may be used to apply a pressure differential along lumen L in order to propel tool 100 in the direction D. In some situations, the use of the pressure differential to propel tool 100 along lumen L may be less time consuming and easier than manually pulling or pushing tool 100 through lumen L. The use of a pressurized fluid to propel tool 100 may also provide additional rinsing of lumen L and may promote further removal of contaminants from lumen L.

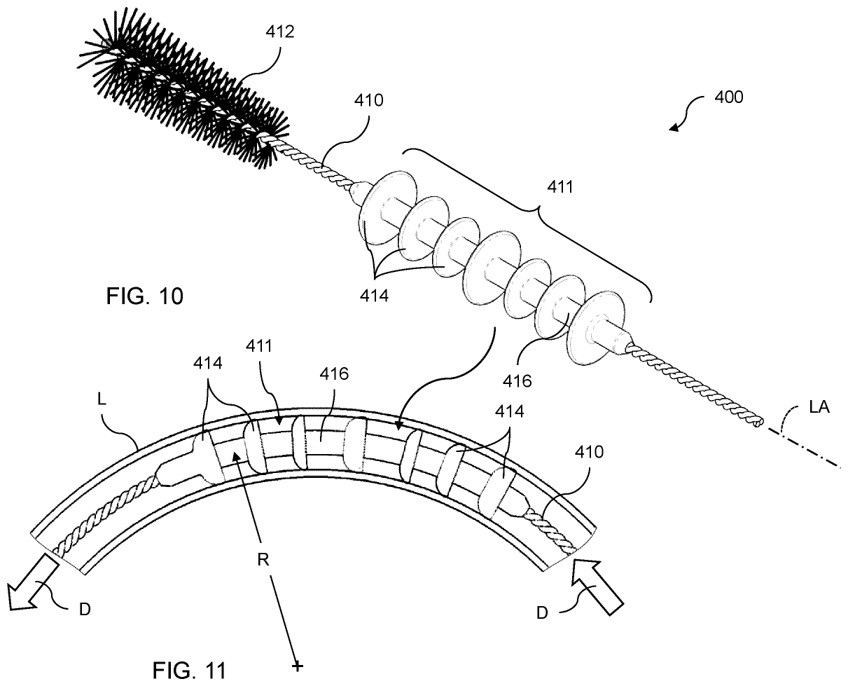

FIG. 10 is a perspective view of another exemplary lumen cleaning tool 400 (referred hereinafter as “tool 400”) for cleaning lumens L (shown in FIG. 11) of medical devices. Tool 400 may have components previously described in relation to other tools described herein. Tool 400 may include wiper 411 and scrubber 412 that are attached to elongated core 410 (referred hereinafter as “core 410”) and disposed at different axial locations along core 410. Core 410 may have longitudinal axis LA. Scrubber 412 may be a bristle brush as described above. Wiper 411 may include base 416 and one or more squeegees 414 in the form of disc-shaped fins extending radially outwardly from base 416. Squeegees 414 may be axially spaced apart along longitudinal axis LA. Squeegees 414 may all have the same size and shape. Alternatively, wiper 411 may include squeegees 414 of different sizes and/or shapes. As explained below, wiper 411 may be interlocked with core 410 along longitudinal axis LA of core 410 using one or more anchors that hinder relative movement between core 410 and wiper 411 along longitudinal axis LA.

FIG. 11 is a side view of part of tool 400 of FIG. 10 disposed inside a curved lumen L of a medical device and being propelled along the lumen L in direction D. In some embodiments, tool 400 may be sufficiently flexible (compliant) to accommodate a desired bend radius R of lumen L. The type(s) and configuration(s) of anchor(s) used to interlock wiper 411 with core 410 may be selected to allow flexibility of tool 400 to permit tool 400 to be inserted into and be propelled through curved lumen L for example. When tool 400 is propelled through lumen L, squeegees 414 of wiper 11 may contact the wall(s) of lumen L and deform while frictionally engaging the wall(s) of lumen L. The frictional engagement of wiper 411 with lumen L may require that the attachment of wiper 411 to core 410 be able to withstand a suitable axial force between wiper 411 and lumen L to prevent wiper 411 from inadvertently becoming dislodged from core 410. Approaches for providing interlocking between various types of lumen interacting bodies and elongated cores are described below.

FIG. 12 is a schematic illustration of an exemplary wiper 411 of tool 400 of FIG. 10 being released from mold parts 418A, 418B where wiper 411 is illustrated as being transparent to show core 410 disposed inside wiper 411. In some embodiments, wiper 411 (or other type of lumen interacting body) may be formed by way of injection molding (e.g., overmolding) onto elongated core 10. However, it is understood that other methods of forming and attaching wiper 411 (or other type of lumen interacting body) onto core 410 may be used. For example, wiper 411 may be made from a relatively soft material that allows wiper 411 to deform and slide along core 410 and pulled or pushed over anchors to engage with (e.g., at least partially encapsulate) anchors that are attached to core 410. In some embodiments, wiper 411 may be formed using a dipping process, a potting process or 3D printing for example. In some embodiments, wiper 411 may be pre-molded into a shape configured to interlock with anchors and then assembled onto core 410 in one or more pieces.

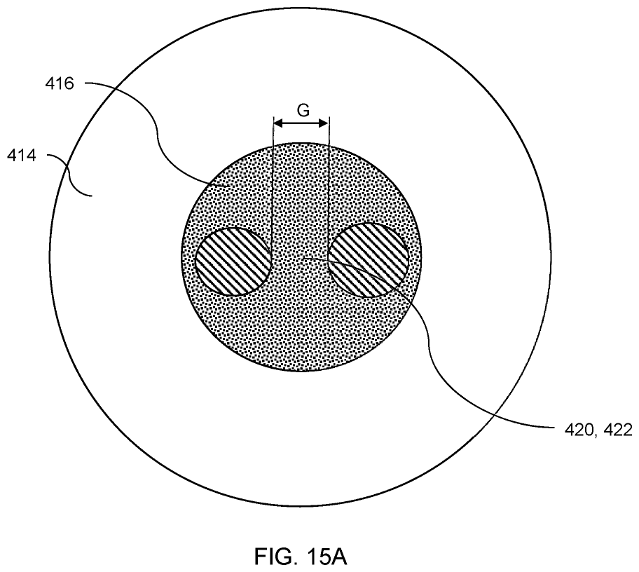

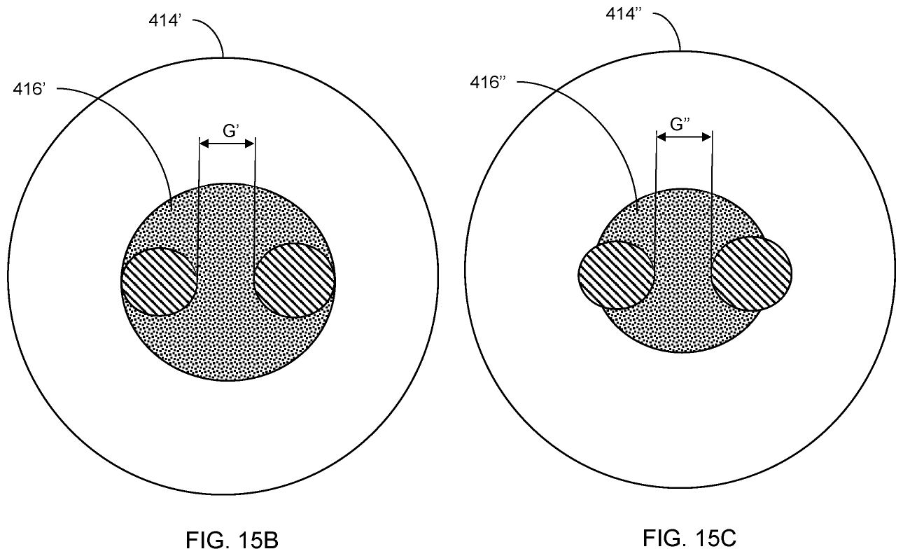

In reference to FIG. 12, overmolding is an injection molding process where one material of wiper 411 such as a TPE for example is molded onto a different material of core 410. The overmolded material of wiper 411 may form an adhesive bond with core 410. The bond between wiper 411 and core 410 may be enhanced by way of one or more anchors interlocking wiper 411 with core 410. Such anchors may include one more beams 420 of overmolding material extending into passages 422 formed through core 410. In the embodiment shown in FIG. 12, core 410 may be defined by (e.g., a pair of) metallic wires twisted (intertwined) together. Core 410 may include first tightly twisted section 410A, loosely twisted section 410B and second tightly twisted section 410C. Sections 410A, 410B, 410C may have different axial positions along core 410. For example, loosely twisted section 410B may be disposed axially between first tightly twisted section 410A and second tightly twisted section 410C. Loosely twisted section 410B may define one or more gaps G (shown in FIGS. 15A-15C) between the wires twisted together and such gaps G may define respective passages 422 extending radially through core 410.

During the overmolding process, mold parts 418A, 418B (e.g., halves) may be closed and brought together over core 410 extending into the cavity defined by mold parts 418A, 418B defining the outer geometry of wiper 411. With mold parts 418A, 418B closed together, overmolding material in the form of a liquidous resin heated above its glass transition temperature for example may be injected into the cavity defined by mold parts 418A, 418B via sprue 419. The overmolding material may fill passages 422 and consequently define beams 420 extending radially through core 410 and interlocking wiper 411 with core 410. After filling the mold cavity defining the shape of wiper 411, and also after cooling and solidification of the overmolding material, mold parts 418A, 418B may be opened to release core 410 and wiper 411 from mold parts 418A, 418B.

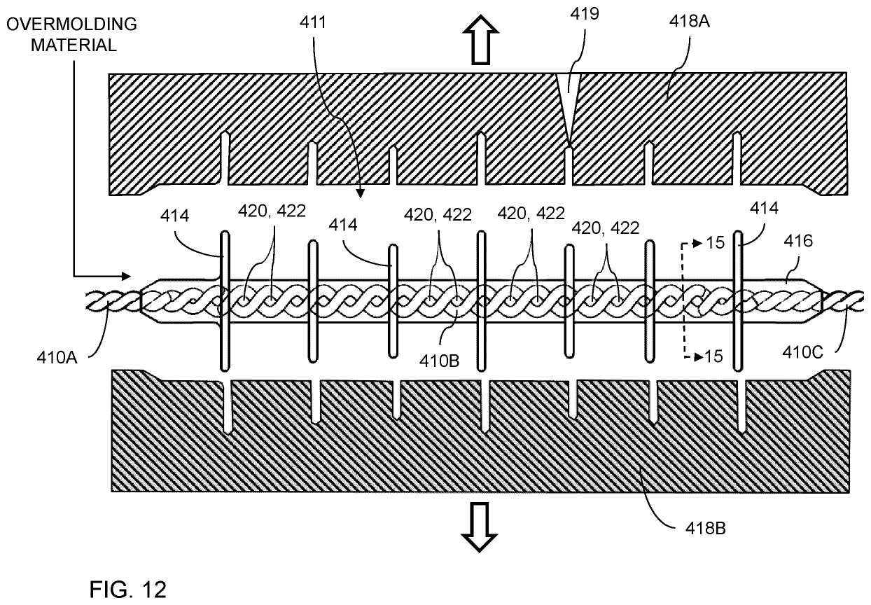

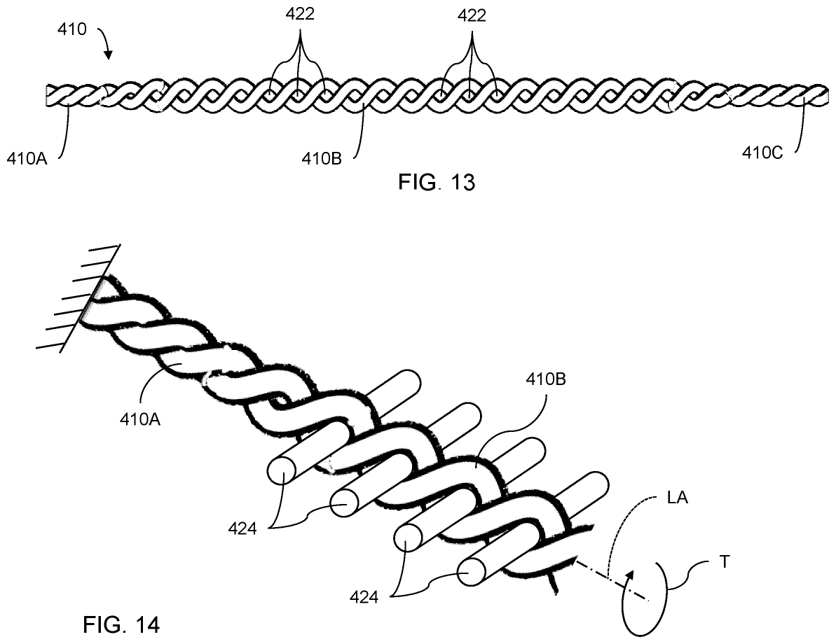

FIG. 13 is a side view of part of core 410 of tool 400 of FIG. 10 shown in isolation. Loosely twisted section 410B, axially disposed between tightly twisted section 410A and second tightly twisted section 410C, may define one or more through passages 422 extending radially through core 410. Even though FIG. 13 shows core 410 being made from wires twisted together, it is understood that core 410 could be have a different construction such as a single wire, a tube, a rod, or a catheter having one or more passages 422 extending (e.g., drilled) partially or completely therethrough for receiving overmolding material and defining one or more respective beams 420.

FIG. 14 is a schematic perspective view of part of core 410 of FIG. 13 during manufacturing of core 410. During the twisting of the metallic wires of core 410 as illustrated by arrow T, one or more pins 424 or other suitable spacers maybe inserted between the wires to form passages 422 in loosely twisted section 410B, and subsequently removed from core 410 in order to define respective passages 422. Another approach for producing loosely twisted section 410B may be to twist the metallic wires of core 410 tightly without the use of pins 424, and then partially untwisting part of core 410 to define loosely twisted section 410B including passages 422.

FIG. 15A is a cross-sectional view of tool 400 of FIG. 12 taken along line 15-15 in FIG. 12. The wires of elongated core 10 may be disposed in base 416 of wiper 411 (or of other type of lumen interacting body). The wires may be fully or partially encapsulated by base 416 of wiper 411. Gap G between the wires may define though passage 422 extending transversely through core 410 and which gets filled with overmolding material to define beam 420. In some embodiments, such gap(s) G may have a dimension of at least 0.25 mm. In some embodiments, gap(s) G may have a dimension of between 1 mm and 2 mm. In some embodiments, gap(s) G may have a dimension of between 1 mm and 8 mm. In some embodiments, gap(s) G may have a dimension of between 6 mm and 8 mm.

Beam 420 may be made of the same overmolding material as wiper 411 and may be connected at opposite ends to base 416. In other words, beam 420 may interconnect portions of wiper 411 disposed on opposite sides of core 410. Accordingly, beams 420 and wiper 411 may have a monolithic construction. It is understood that the overmolding of beams 420 may also be used with other types of overmolded lumen interacting bodies such as lumen stabilizers for example.

FIGS. 15B and 15C are cross-sectional views of other lumen cleaning tools 414′, 414″ of the type shown in FIG. 12 taken along line 15-15 of FIG. 12. As shown in FIG. 15B, in some embodiments, the wires of elongated core 10 may be disposed in base 416′ of wiper 411′, spaced apart by gap G′ and may be flush with a radially outer surface of base 416′. As shown in FIG. 15C, in some embodiments, the wires of elongated core 10 may be disposed in base 416″ of wiper 411″, spaced apart by gap G″ and may protrude out of the radially outer surface of base 416″.

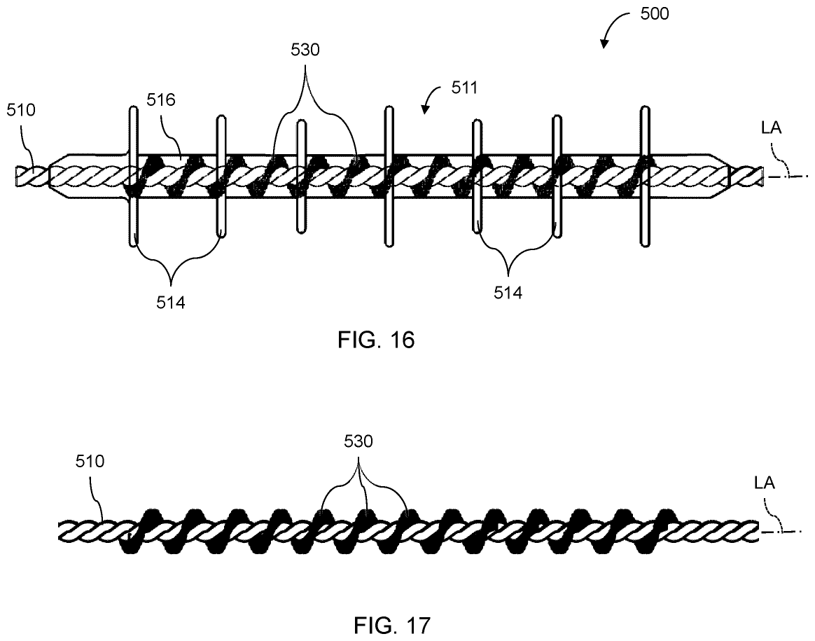

FIG. 16 is a side view of another exemplary lumen cleaning tool 500 (referred hereinafter as “tool 500”) for cleaning lumens L (shown in FIG. 11) of medical devices. FIG. 17 is a side view of elongated core 510 (referred hereinafter as “core 510”) and bristles 530 of tool 500. Tool 500 may have components previously described in relation to other tools described herein. In reference to FIGS. 16 and 17, tool 500 may include wiper 511 and/or one or more other lumen interacting bodies attached to core 510 and disposed at different axial location(s) along core 510. Wiper 511 may include base 516 and one or more squeegees 514 in the form of disc-shaped fins extending radially outwardly from base 516. Wiper 511 may be interlocked with core 510 along longitudinal axis LA of core 510 using one or more fibrous anchors in the form of one or more bristles 530 that hinder relative movement between core 510 and wiper 511 along longitudinal axis LA.

Wiper 511 is illustrated as being transparent to show core 510 with bristles 530 attached to core 510 and being at least partially encapsulated by wiper 511. Bristles 530 may serve as protuberances attached to core 510 and interlocking wiper 511 with core 510. Bristles 530 may be inserted between the wires and then the wires may be twisted together to press (clamp) and retain bristles 530 therebetween when core 510 is manufactured. Other suitable methods of attaching bristles 530 to core 510 may be used. Wiper 511 may be overmolded onto core 510 and bristles 530 so that bristles 530 may be at least partially encapsulated by the overmolding material of wiper 511. Bristles 530 may be made of a suitable polymer.

In some embodiments, bristles 530 may be relatively short so as not to protrude radially outwardly from base 516. For example, bristles 530 may remain concealed inside wiper 511 and not interfere with the function of wiper 511. For example, relative to longitudinal axis LA, bristles 530 may have a radial dimension that is the same or smaller than a radial dimension of base 516. In some embodiments, bristles 530 may extend radially outwardly (i.e., protrude) from the core 510 and may be arranged to define a helical arrangement that extends along longitudinal axis LA. The helical arrangement of bristles 530 may extend along longitudinal axis LA by an amount that covers at least a majority of the axial span of wiper 511 for example. It is understood that other types of anchor(s) defining a helical shape may be attached (e.g., glued, welded, crimped, soldered, brazed, molded or frictionally engaged) to core 510 for the purpose of interlocking wiper 511 with core 510.

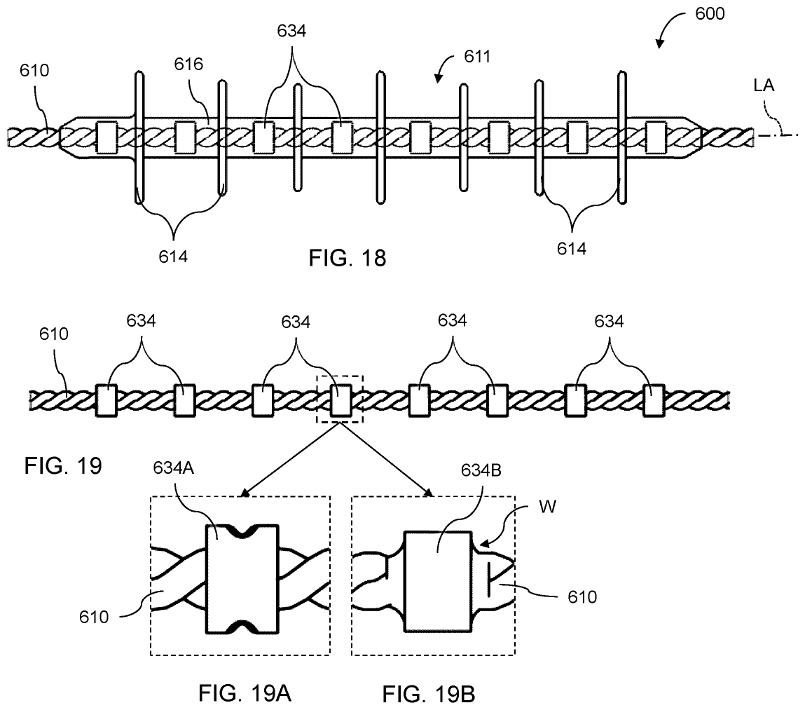

FIG. 18 is a side view of another exemplary lumen cleaning tool 600 (referred hereinafter as “tool 600”) for cleaning lumens L (shown in FIG. 11) of medical devices. FIG. 19 is a side view of elongated core 610 (referred hereinafter as “core 610”) and ring-shaped anchors 634 of tool 600. Tool 600 may have components previously described in relation to other tools described herein. In reference to FIGS. 18 and 19, tool 600 may include wiper 611 and/or one or more other lumen interacting bodies attached to core 610 and disposed at different axial location(s) along core 610. Wiper 611 may include base 616 and one or more squeegees 614 in the form of disc-shaped fins extending radially outwardly from base 616. Wiper 611 may be interlocked with core 610 along longitudinal axis LA of core 610 using one or more ring-shaped anchors 634 attached to core 610 and that hinder relative movement between core 610 and wiper 611 along longitudinal axis LA.

Wiper 611 is illustrated as being transparent to show core 610 with ring-shaped anchors 634 attached to core 610 and being at least partially encapsulated by wiper 611. Ring-shaped anchors 634 may serve as protuberances attached to core 610 and interlocking wiper 611 with core 610. Wiper 611 may be overmolded onto core 610 and ring-shaped anchors 634 so that ring-shaped anchors 634 may be at least partially encapsulated by the overmolding material of wiper 611. In embodiments where a plurality of ring-shaped anchors 634 are used, ring-shaped anchors 634 may configured and spaced apart to permit some flexibility of tool 600 to accommodate curved lumens L. For example, ring-shaped anchors 634 may be selected to have relatively short axial lengths and also be spaced apart to avoid interference with each other when core 610 is bent to accommodate a desired bend radius R (shown in FIG. 11). For example, relative to longitudinal axis LA, ring-shaped anchors 634 may have a radial dimension that is the same or smaller than a radial dimension of base 616 so as not to protrude radially outwardly from base 616. For example, ring-shaped anchors 634 may remain concealed inside wiper 611 and not interfere with the function of wiper 611.

FIG. 19A is a magnified side view of an exemplary metallic ring-shaped anchor 634A being attached to core 610 by way of crimping. During assembly, core 610 may be inserted through a central opening of ring-shaped anchor(s) 634A and ring-shaped anchor(s) 634A may be slid to their desired axial locations along core 610. Ring-shaped anchor(s) 634A may then be plastically deformed (e.g., crimped) onto core 610.

FIG. 19B is a magnified side view of an exemplary metallic ring-shaped anchor 634B being attached to core 610 by way of welding via weld W. During assembly, core 610 may be inserted through a central opening of ring-shaped anchor(s) 634B and ring-shaped anchor(s) 634B may be slid to their desired axial locations along core 610. Ring-shaped anchor(s) 634A may then be laser welded onto core 610.

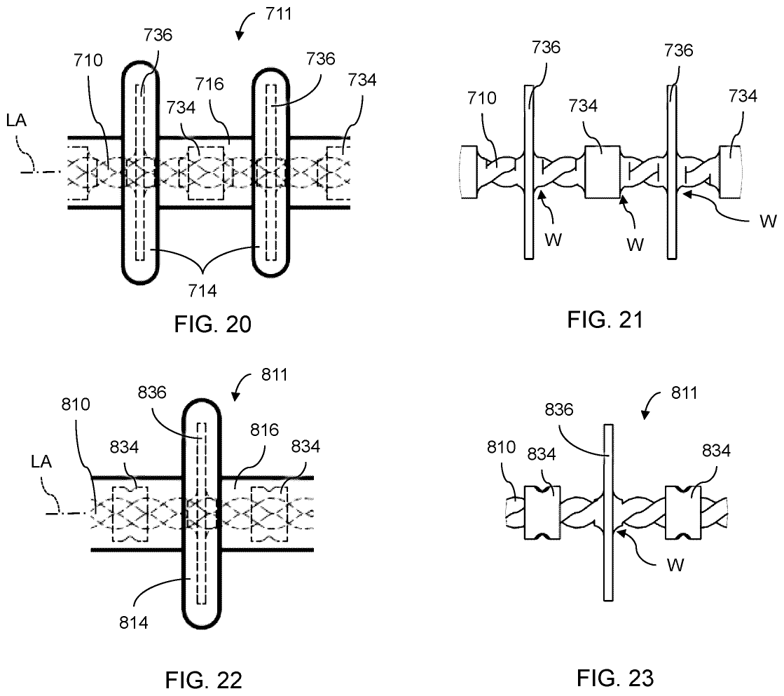

FIG. 20 is a side view of part of another exemplary lumen cleaning tool for cleaning lumens L (shown in FIG. 11) of medical devices. FIG. 21 is a side view of elongated core 710 (referred hereinafter as “core 710”), ring-shaped anchors 734 and disc-shaped anchors 736. In reference to FIG. 21, ring-shaped anchor(s) 734 and disc-shaped anchor(s) 736 may each be welded to core 710 via welds W. The tool of FIG. 20 may include components previously described in relation to other tools described herein. In reference to FIGS. 20 and 21, wiper 711 may include base 716 and one or more squeegees 714 in the form of disc-shaped fins extending radially outwardly from base 716. Wiper 711 may be interlocked with core 710 along longitudinal axis LA of core 710 using one or more ring-shaped anchors 734 and disc-shaped anchors 736 attached to core 710 and that hinder relative movement between core 710 and wiper 711 along longitudinal axis LA.

Wiper 711 is illustrated as being transparent to show, in broken lines, core 710 with ring-shaped anchors 734 and disc-shaped anchors 736 attached to core 710 and being at least partially encapsulated by wiper 711. Ring-shaped anchors 734 and disc-shaped anchors 736 may serve as protuberances attached to core 710 and interlocking wiper 711 with core 710. Wiper 711 may be overmolded onto core 710, ring-shaped anchors 734 and disc-shaped anchors 736 so that ring-shaped anchors 734 and disc-shaped anchors 736 may be at least partially encapsulated by the overmolding material of wiper 711. In embodiments where a plurality of ring-shaped anchors 734 and disc-shaped anchors 736 are used, ring-shaped anchors 734 and disc-shaped anchors 736 may configured and spaced apart to permit some flexibility of the tool to accommodate curved lumens L.

Relative to longitudinal axis LA, ring-shaped anchor(s) 734 may have a radial dimension that is the same or smaller than a radial dimension of base 716 so as not to protrude radially outwardly from base 716. Ring-shaped anchor(s) 734 may be located axially between neighbouring squeegees 714. Disc-shaped anchor(s) 736 may have a radial dimension that is greater than that of ring-shaped anchor(s) 734. Disc-shaped anchor(s) 736 may have a radial dimension that is greater than a radial dimension of base 716. Disc-shaped anchors 736 may be located axially coincident with squeegees 714 and may occupy space inside a radially-extending fin of wiper 711 for example. In some embodiments, disc-shaped anchors 736 may be made from a harder material than the overmolding material of wiper 711 and may therefore serve to stiffen squeegees 714 or otherwise influence the rigidity or other mechanical properties of wiper 711. For example, disc-shaped anchors 736 or other anchors may modify mechanical properties of the relatively soft wiper 711 (or other lumen interacting member) to tailor rigidity, flexibility or malleability, as may be desired for a specific application, while the relatively soft wiper 711 may provide atraumatic engagement with the lumen L. In some embodiments, ring-shaped anchor(s) 734 and disc-shaped anchor(s) 736 may be disposed in an alternating manner along core 710.

FIG. 22 is a side view of part of another exemplary lumen cleaning tool for cleaning lumens L (shown in FIG. 11) of medical devices. FIG. 23 is a side view of elongated core 810 (referred hereinafter as “core 810”), ring-shaped anchors 834 and disc-shaped anchors 836. In contrast with the tool shown in FIGS. 20 and 21, ring-shaped anchor(s) 734 may be crimped onto core 810 and disc-shaped anchor(s) 836 may be welded to core 810 via weld W. The tool of FIG. 22 may include components previously described in relation to other tools described herein. In reference to FIGS. 22 and 23, wiper 811 may include base 816 and one or more squeegees 814 in the form of disc-shaped fins extending radially outwardly from base 816. Wiper 811 may be interlocked with core 810 along longitudinal axis LA of core 810 using one or more ring-shaped anchors 834 and disc-shaped anchors 836 attached to core 810 and that hinder relative movement between core 810 and wiper 811 along longitudinal axis LA. Wiper 811 is illustrated as being transparent to show, in broken lines, core 810 with ring-shaped anchors 834 and disc-shaped anchors 836 attached to core 810 and being at least partially encapsulated by wiper 811.

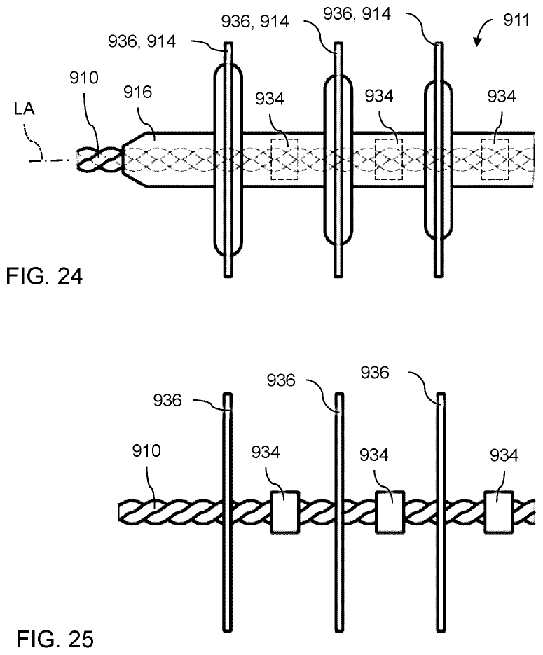

FIG. 24 is a side view of part of another exemplary lumen cleaning tool for cleaning lumens L (shown in FIG. 11) of medical devices. FIG. 25 is a side view of elongated core 910 (referred hereinafter as “core 910”), ring-shaped anchors 934 and disc-shaped anchors 936. In contrast with the tool shown in FIGS. 20 and 21, disc-shaped anchor(s) 936 may extend radially outwardly beyond the overmolding material of wiper 911. In other words, disc-shaped anchors 936 may define protuberances that protrude out of overmolded body of wiper 911. For example, disc-shaped anchors 936 may interact directly with lumen L during use. The tool of FIG. 24 may include components previously described in relation to other tools described herein. In reference to FIGS. 24 and 25, wiper 911 may include base 916 and one or more squeegees 914 in the form of disc-shaped fins extending radially outwardly from base 916. Wiper 911 may be interlocked with core 910 along longitudinal axis LA of core 910 using one or more ring-shaped anchors 934 and disc-shaped anchors 936 attached to core 910 and that hinder relative movement between core 910 and wiper 911 along longitudinal axis LA. Wiper 911 is illustrated as being transparent to show, in broken lines, core 910 with ring-shaped anchors 934 and, in solid lines, disc-shaped anchors 936 attached to core 910 and being at least partially encapsulated by wiper 911.

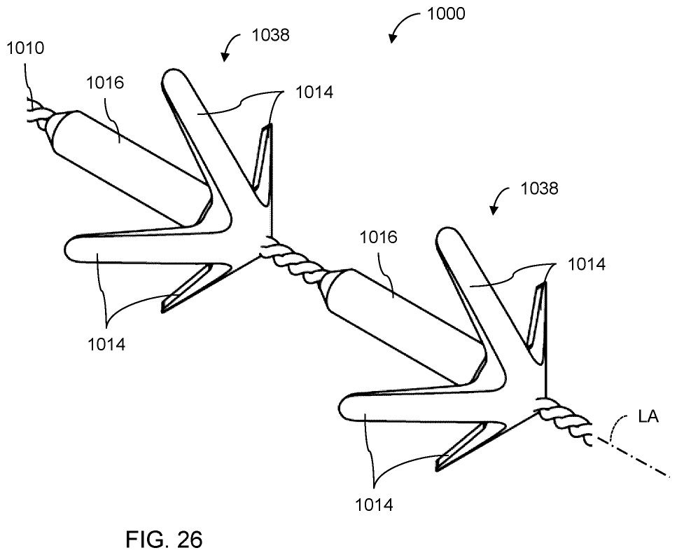

FIG. 26 is a perspective view of an exemplary lumen stabilizing tool 1000 (referred hereinafter as “tool 1000”) for stabilizing an anatomical or artificial lumen. Tool 1000 may include one or more lumen stabilizers 1038 (referred hereinafter in the singular) and/or other type of lumen interacting body(ies) attached to elongated core 1010 (referred hereinafter as “core 1010”) and disposed at different axial locations along core 1010. Core 1010 may have longitudinal axis LA. Lumen stabilizer 1038 may have an umbrella-like structure suitable for holding the lumen open without completely occluding the lumen so that fluid flow may be permitted along the lumen. Lumen stabilizer 1038 may include base 1016 and one or more arms 1014 extending radially outward from base 1016. Each arm may include a proximal end attached to base 1016 and a distal free end located radially outwardly of base 1016 and of core 1010. Lumen stabilizers 1038 may be interlocked with core 1010 along longitudinal axis LA of core 1010 using one or more anchors that hinder relative movement between core 1010 and lumen stabilizers 1038 along longitudinal axis LA.

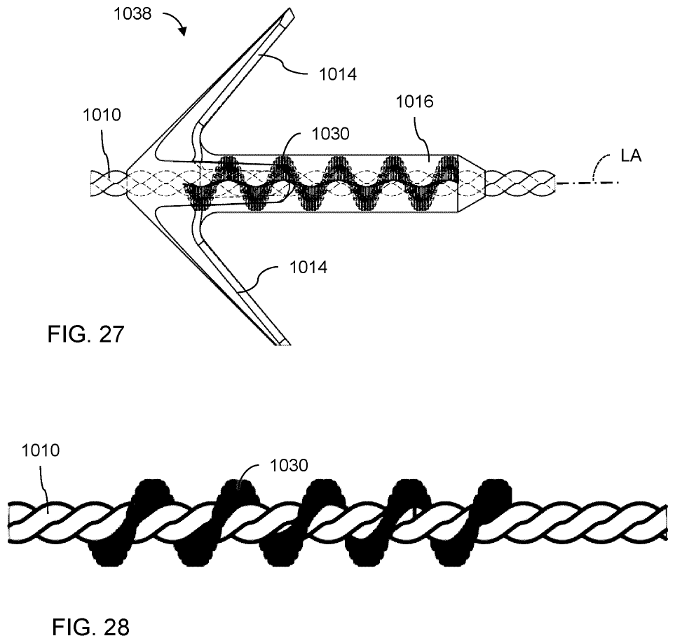

FIG. 27 is a side view of part of tool 1000 where lumen stabilizer 1038 is illustrated as being transparent to show core 1010 with bristles 1030 attached thereto. FIG. 28 is a side view of core 1010 and bristles 1030. As explained above in relation to FIGS. 16 and 17, bristles 1030 may serve as protuberances attached to core 1010 and interlock lumen stabilizer 1038 with core 1010. Bristles 1030 may be inserted between the wires of core 1010 and then the wires may be twisted together to press (clamp) and retain bristles 1030 therebetween when core 1010 is manufactured. Lumen stabilizer 1038 may be overmolded onto core 1010 and bristles 1030 so that bristles 1030 may be at least partially encapsulated by the overmolding material of lumen stabilizer 1038. Bristles 1030 may be dimensioned to remain concealed inside lumen stabilizer 1038. In some embodiments, bristles 1030 may extend radially outwardly (i.e., protrude) from the core 1010 and may be arranged to define a helical arrangement that extends along longitudinal axis LA. The helical arrangement of bristles 1030 may extend along longitudinal axis LA by an amount that covers at least a majority of the axial span of lumen stabilizer 1038 for example.

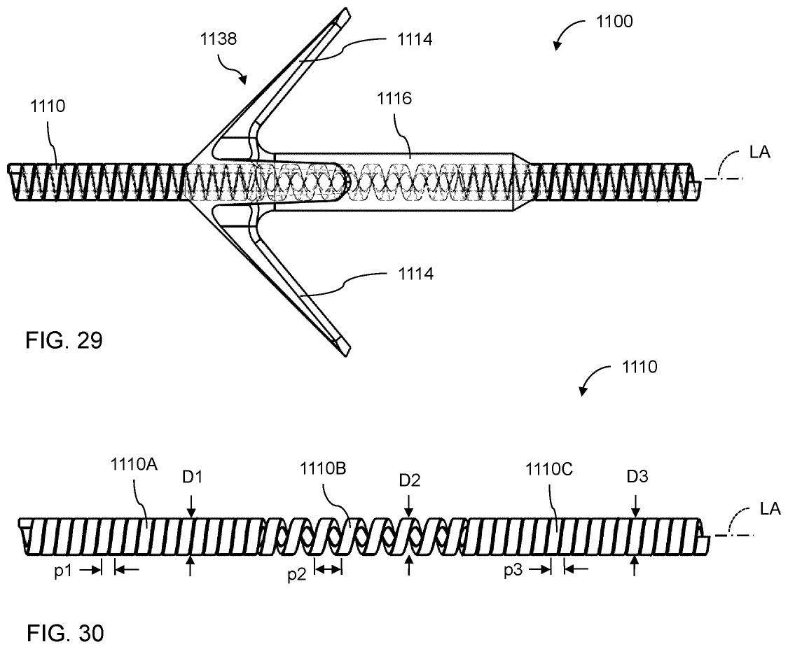

FIG. 29 is a side view of part of another exemplary lumen stabilizing tool 1100 (referred hereinafter as “tool 1100”) for stabilizing an anatomical or artificial lumen. Tool 1100 may include one or more lumen stabilizers 1138 (referred hereinafter in the singular) and/or other type of lumen interacting body(ies) attached to elongated core 1110 (referred hereinafter as “core 1110”) and disposed at different axial locations along core 1110. Core 1110 may have longitudinal axis LA. Lumen stabilizer 1138 is illustrated as being transparent to show core 1110 extending therethrough. Lumen stabilizer 1138 may have an umbrella-like structure suitable for holding the lumen open without completely occluding the lumen. Lumen stabilizer 1138 may include base 1116 and one or more arms 1114 extending radially outward from base 1116. Lumen stabilizer 1138 may be interlocked with core 1110 along longitudinal axis LA of core 1110 using one or more anchors that hinder relative movement between core 1110 and lumen stabilizer 1138 along longitudinal axis LA and may prevent unwanted migration of lumen stabilizer 1138.

FIG. 30 is a side view of core 1110 of tool 1100 of FIG. 29 shown in isolation. Core 1110 may include a helical coil that is made from only one (i.e., a sole) wire, or from multiple wires to facilitate torque transfer by core 1110. For example, the helical coil may be made from one or more metallic or polymeric wires. The helical coil may include first section 1110A having first pitch p1 and second section 1110B having second pitch p2 larger than first pitch p1 so that p2>p1 as shown in FIG. 30. The helical coil may include third section 1110C having third pitch p3. Second pitch p2 may be larger than third pitch p3 so that p2>p3 as shown in FIG. 30. In some embodiments, first pitch p1 and third pitch p3 may be substantially equal. In some embodiments, second pitch p2 may be at least 10% greater than first pitch p1 or third pitch p3. In some embodiments, second pitch p2 may be between 10% and 100% greater than first pitch p1 or third pitch p3.

First section 1110A may be axially adjacent second section 1110B along longitudinal axis LA of the helical coil. Second section 1110B may be axially adjacent third section 1110C along longitudinal axis LA of the helical coil. Second section 1110B may be located between first section 1110A and third section 1110C along longitudinal axis LA of the helical coil. In some embodiments, core 1110 may have a substantially uniform diameter along longitudinal axis LA so that first diameter D1, second diameter D2 and third diameter D3 may be substantially equal. As shown in FIG. 30 as an example, the wire defining the helical coil may have a polygonal (e.g., square, rectangular, quadrilateral, hexagonal) cross-sectional profile. Lumen stabilizer 1138 (or a wiper, or another lumen interacting device) may be attached to the helical coil and anchored to second section 1110C of core 1110 as explained below.

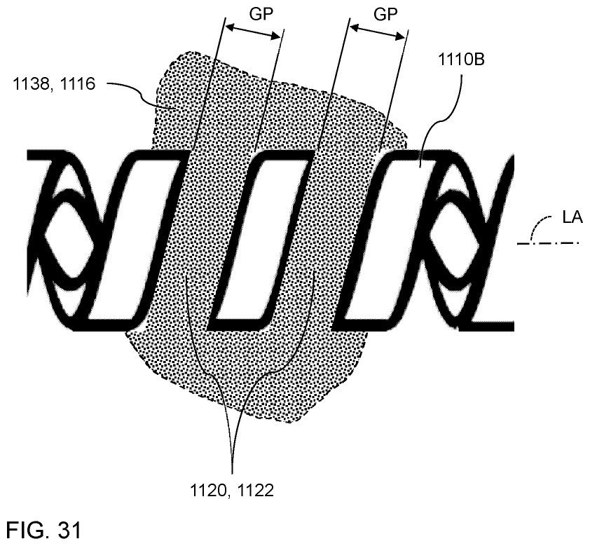

FIG. 31 is an enlarged side view of part of second section 1110B of core 1110 with lumen stabilizer 1138 overmolded thereon so that core 1110 may be at least partially encapsulated by overmolding material. The helical coil may be disposed inside base 1116 of lumen stabilizer 1138. Gaps GP defined between adjacent wire turns may define respective though passages 1122 extending transversely through core 1110 and which get filled with overmolding material to define respective beams 1120. In some embodiments, such gap(s) GP may have a dimension of at least 0.25 mm. In some embodiments, gap(s) GP may have a dimension of between 1 mm and 2 mm. In some embodiments, gap(s) GP may have a dimension of between 1 mm and 8 mm. In some embodiments, gap(s) GP may have a dimension of between 6 mm and 8 mm. Beams 1120 may be made of the same overmolding material as lumen stabilizer 1138 and may be connected at opposite ends to base 1116. In other words, beams 1120 may each interconnect portions of lumen stabilizer 1138 disposed on opposite sides of core 1110. Accordingly, beams 1120 and base 1116 may have a monolithic construction.

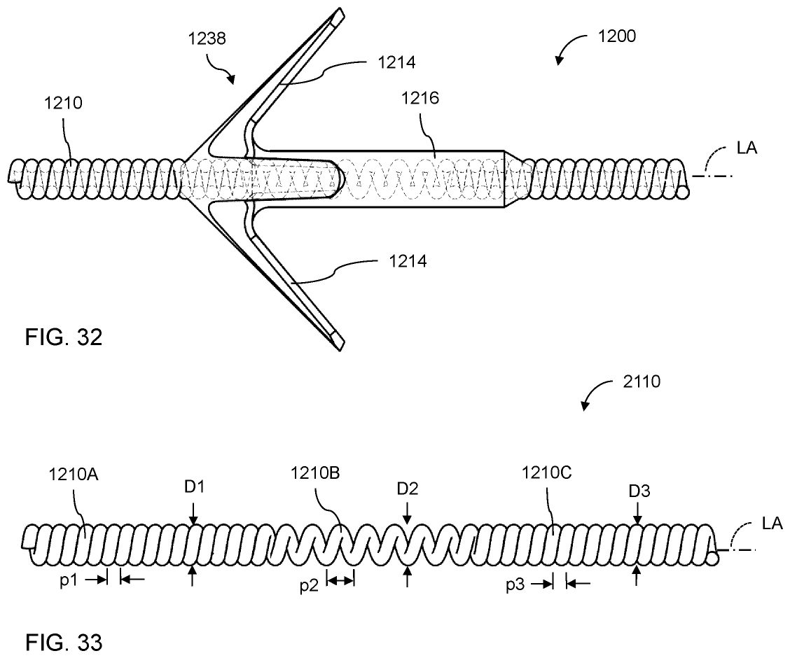

FIG. 32 is a side view of part of another exemplary lumen stabilizing tool 1200 (referred hereinafter as “tool 1200”) for stabilizing an anatomical or artificial lumen. Tool 1200 may include one or more lumen stabilizers 1238 (referred hereinafter in the singular) and/or other type of lumen interacting body(ies) attached to elongated core 1210 (referred hereinafter as “core 1210”) and disposed at different axial locations along core 1210. Core 1210 may have longitudinal axis LA. Lumen stabilizer 1238 is illustrated as being transparent to show core 1110 extending therethrough. Lumen stabilizer 1238 may include base 1216 and one or more arms 1214 extending radially outward from base 1216. Lumen stabilizer 1238 may be interlocked with core 1210 along longitudinal axis LA of core 1210 using one or more anchors that hinder relative movement between core 1210 and lumen stabilizer 1238 along longitudinal axis LA.

FIG. 33 is a side view of core 1210 of tool 1200 of FIG. 32 shown in isolation. Core 1210 may include a helical coil that is made from only one (i.e., a sole) wire, or from multiple wires to facilitate torque transfer by core 1210. As shown in FIG. 33 the wire defining the helical coil may have a circular cross-sectional profile. However, the wire may have instead have an oval, semicircular, oblong or polygonal cross-sectional profile. The wire may have a cross-sectional profile that includes a combination of straight/flat and curved/rounded segments. The wire may have a uniform or non-uniform cross-sectional profile along its length. For example, the wire may vary in size and/or shape along its length. The helical coil may include first section 1210A having first pitch p1 and second section 1210B having second pitch p2 larger than first pitch p1 so that p2>p1. The helical coil may include third section 1210C having third pitch p3. Second pitch p2 may be larger than third pitch p3 so that p2>p3. In some embodiments, first pitch p1 and third pitch p3 may be substantially equal. In some embodiments, second pitch p2 may be at least 10% greater than first pitch p1 or third pitch p3. In some embodiments, second pitch p2 may be between 10% and 100% greater than first pitch p1 or third pitch p3.

In some embodiments, core 1210 may have a substantially uniform diameter along longitudinal axis LA so that first diameter D1, second diameter D2 and third diameter D3 may be substantially equal. Lumen stabilizer 1238 (or a wiper, or another lumen interacting device) may be attached to the helical coil and anchored to second section 1210C of core 1210 in substantially the same manner as shown in FIG. 31.

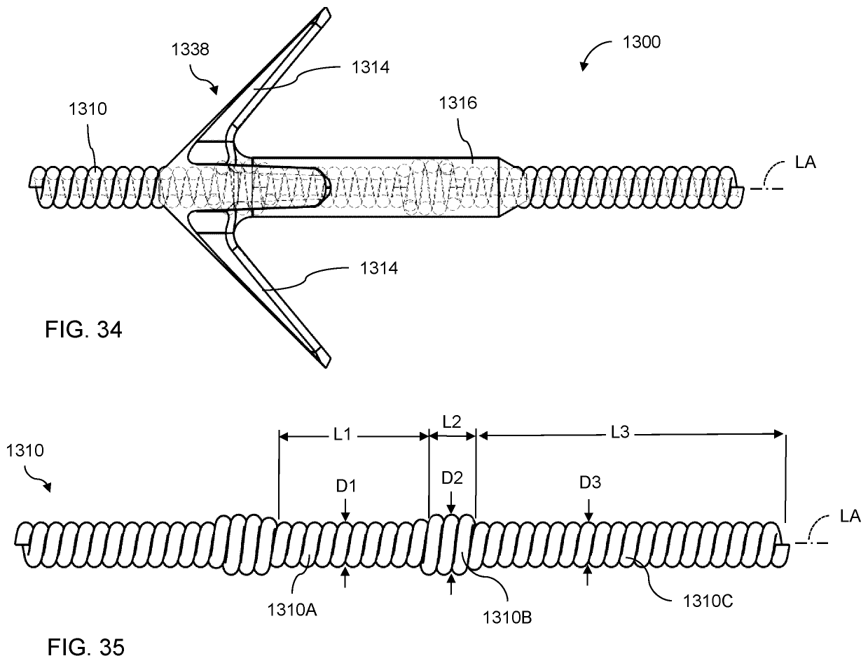

FIG. 34 is a side view of part of another exemplary lumen stabilizing tool 1300 (referred hereinafter as “tool 1300”) for stabilizing an anatomical or artificial lumen. Tool 1300 may include one or more lumen stabilizers 1338 (referred hereinafter in the singular) and/or other type of lumen interacting body(ies) attached to elongated core 1310 (referred hereinafter as “core 1310”) and disposed at different axial locations along core 1310. Core 1310 may have longitudinal axis LA. Lumen stabilizer 1338 is illustrated as being transparent to show core 1310 extending therethrough. Lumen stabilizer 1338 may include base 1316 and one or more arms 1314 extending radially outward from base 1316. Lumen stabilizer 1338 may be interlocked with core 1310 along longitudinal axis LA of core 1310 using one or more anchors that hinder relative movement between core 1310 and lumen stabilizer 1338 along longitudinal axis LA.

FIG. 35 is a side view of core 1310 of tool 1300 of FIG. 32 shown in isolation. Core 1310 may include a helical coil that is made from only one (i.e., a sole) wire, or from multiple wires to facilitate torque transfer by core 1310. The helical coil may include first section 1310A having first outer diameter D1 and second section 1310B having second outer diameter D2 larger than first outer diameter D1 so that D2>D1. The helical coil may include third section 1310C having third outer diameter D3. Second outer diameter D2 may be larger than third outer diameter D3 so that D2>D3. In some embodiments, first outer diameter D1 and third outer diameter D3 may be substantially equal. First outer diameter D1 and third outer diameter D3 may define a nominal outer diameter of core 1310. Second section 1310B may have an increased outer diameter D2 relative to a nominal diameter of core 1310. In some embodiments, second outer diameter D2 may be at least 10% greater than first outer diameter D1 or third outer diameter D3. In some embodiments, second outer diameter D2 may be between 10% and 100% greater than first outer diameter D1 or third outer diameter D3. Second section 1310B may be located axially between first section 1310A and third section 1310B.

Lumen stabilizer 1338 (or a wiper, or another lumen interacting device) may be attached to the helical coil and anchored to second section 1310C of increased outer diameter D2 of core 1310. In other words, second section 1310C may define a protuberance attached to core 1310 for interlocking lumen stabilizer 1338 with core 1310. In some embodiments, lumen stabilizer 1338 may be overmolded onto core 1310 and encapsulate core 1310. In some embodiments, core 1310 may include one or more sections of increased diameter serving as protuberances for interlocking lumen stabilizer 1338 with core 1310. In some embodiments, second section 1310B may have axial span L2 that is smaller than axial span L1 of first section 1310A. Second section 1310B may have axial span L2 that is smaller than axial span L3 of third section 1310C.

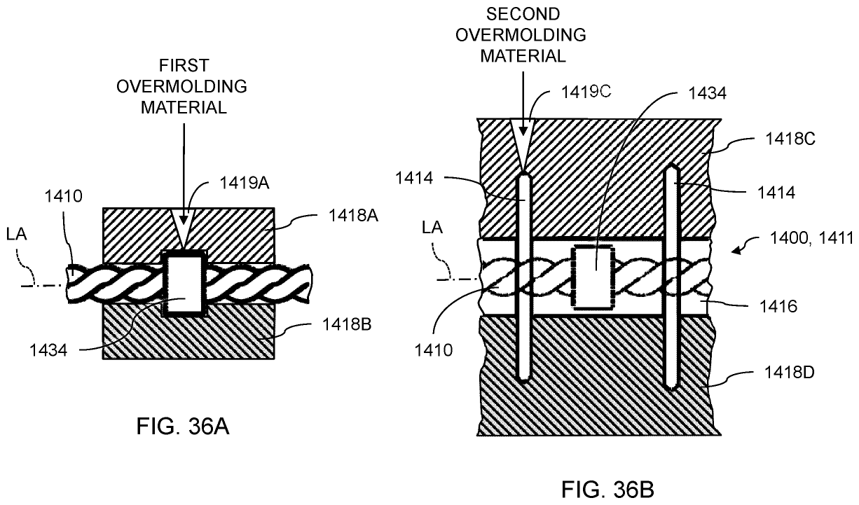

FIGS. 36A and 36B schematically illustrate an exemplary two-shot overmolding method for manufacturing an exemplary lumen cleaning tool 1400 (referred hereinafter as “tool 1400”) for cleaning lumens L (shown in FIG. 11) of medical devices, or for manufacturing another type of lumen interacting tool. Tool 1400 may include elongated core 1410 (referred hereinafter as “core 1410”), which may include twisted metallic wires, one or more anchors 1434 (referred hereinafter in the singular), and one or more wipers 1411 (referred hereinafter in the singular). Tool 1400 may have components previously described in relation to other tools described herein. Tool 1400 may include one or more other lumen interacting bodies (e.g., lumen stabilizers) attached to core 1410 and disposed at different axial location(s) along core 1410. Wiper 1411 may include base 1416 and one or more squeegees 1414 in the form of disc-shaped fins extending radially outwardly from base 1416. Wiper 1411 may be interlocked with core 1410 along longitudinal axis LA of core 1410 using anchor 1434 attached to core 1410 and hindering relative movement between core 1410 and wiper 1411 along longitudinal axis LA.

Anchor 1434 and wiper 1411 may both be produced by overmolding using different overmolding materials providing suitable melt adhesion therebetween. In some embodiments, anchor 1434 and wiper 1411 may be produced using a two-shot overmolding process in which one material is injection molded over a previously injection molded substrate. For example, in reference to FIG. 36A, anchor 1434 may first be overmolded onto core 1410 using a first overmolding material. Mold parts 1418A, 1418B (e.g., halves) may be brought together and sealed against core 1410 extending into the cavity defined by mold parts 1418A, 1418B defining the outer geometry of anchor 1434. With mold parts 1418A, 1418B closed together, the first overmolding material in the form of a liquidous resin heated above its glass transition temperature for example may be injected into the cavity defined by mold parts 1418A, 1418B via sprue 1419A. The first overmolding material may fill the cavity and consequently form anchor 1434 extending radially outward from core 1410. After filling the mold cavity and cooling of the first overmolding material, mold parts 1418A, 1418B may be opened to release core 1410 and anchor 1434 from mold parts 1418A, 1418B.

In reference to FIG. 36B, wiper 1411 may subsequently be overmolded onto anchor 1434 and onto core 1410 using a second overmolding material different from the first overmolding material. In other words, anchor 1434 and core 1410 may serve as a substrate onto which wiper 1411 may be overmolded. Wiper 1411 is illustrated as being transparent to show core 1410 with anchor 1434 attached to core 1410 and being at least partially encapsulated by wiper 1411. Mold parts 1418C, 1418D (e.g., halves) may be brought together and sealed against core 1410 extending into the cavity defined by mold parts 1418C, 1418D defining the outer geometry of wiper 1411. With mold parts 1418C, 1418D closed together, the second overmolding material in the form of a liquidous resin heated above its glass transition temperature for example may be injected into the cavity defined by mold parts 1418C, 1418D via sprue 1419C. The second overmolding material may fill the cavity and consequently define wiper 1411 encapsulating anchor 1434. After filling the mold cavity and cooling of the first overmolding material, mold parts 1418C, 1418D may be opened to release core 1410 and wiper 1411 from mold parts 1418C, 1418D.

The first overmolding material of anchor(s) 1434 may be of a different type than the second overmolding material used to subsequently form wiper 1411. For example, the first overmolding material may be harder than the second overmolding material. For example, the first overmolding material may provide a better adhesion to core 1410 than the second overmolding material, and may therefore help with hindering the movement of wiper 1411 along core 1410 during use of tool 1400. As an example, the first overmolding material may be polypropylene (PP), and the second overmolding material may be TPE, which may adhere relatively well to PP. Accordingly, anchor(s) 1434 may serve as an interlock in the form of a protuberance obstructing the movement of wiper 1411 along core 1410. Alternatively or in addition, anchor(s) 1434 may hinder the movement of wiper 1411 by way of the (e.g., melt) adhesion between the first overmolding material and the second overmolding material.

It is understood that the two-shot overmolding method illustrated in FIGS. 36A and 36B is exemplary only and other methods may be used to form anchor(s) 1434 and/or wiper 1411. For example, anchor(s) 1434 could instead be deposited by brushing on, dipping, plating, or other method prior to overmolding of wiper 1411.

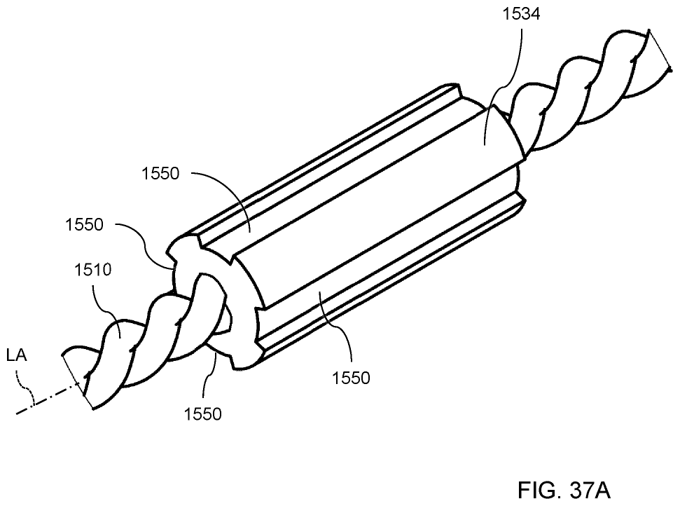

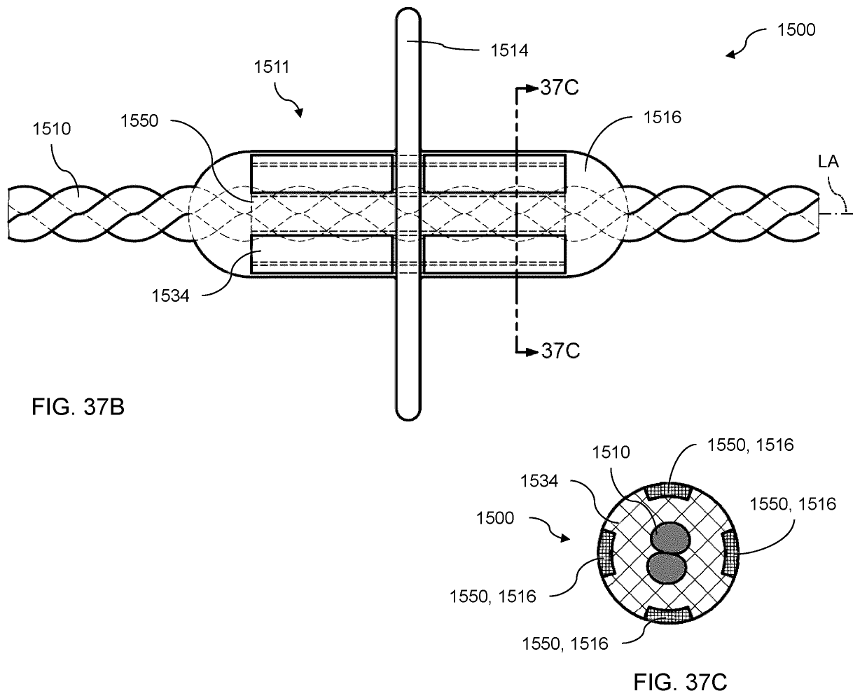

FIG. 37A is a perspective view of another exemplary anchor 1534 formed by overmolding as shown in FIG. 36A or by another method. Anchor 1534 may have any suitable shape and may have a relatively smooth outer surface for adhering to the second material of the lumen interacting body such as wiper 1511 (shown in FIG. 37B) that may at least partially encapsulate anchor 1534. Alternatively, anchor 1534 may define one or more features that enhance the grip between anchor 1534 and wiper 1511. Such features may include a surface texture, ribs, circumferential and/or longitudinal grooves 1550, protrusions, recesses, voids or other surface irregularities that may promote the melt adhesion by increasing the contact surface area between the first material of anchor 1534 and the second material of wiper 1511.

FIG. 37B is a side view of an exemplary lumen interacting tool 1500 including a lumen interacting body such as wiper 1511 overmolded or otherwise deposited onto anchor 1534 of FIG. 37A. Wiper 1511 may include base 1516 and squeegee 1514 extending radially outwardly from base 1516. In some embodiments, tool 1500 may be manufactured using the two-shot overmolding method illustrated in FIGS. 36A and 36B. Anchor 1534 is shown in broken lines and may include a protuberance that is only partially encapsulated by base 1516 of wiper 1511. In other embodiments, anchor 1534 may be fully encapsulated by base 1516.

FIG. 37C is a cross-sectional view of tool 1500 taken along line 37C-37C in FIG. 37B. The overmolding material of wiper 1511 may occupy grooves 1550 and provide a suitable melt adhesion with the material of anchor 1534. In addition, the cooperating geometries of anchor 1534 and wiper 1511 may fit into each other to also provide mechanical interlocking between anchor 1534 and wiper 1511. As shown in FIG. 37C, anchor 1534 may be only partially encapsulated by base 1516 of wiper 1511 so that parts of anchor 1534 that are between grooves 1550 may be exposed to the exterior of tool 1500. In some embodiments, a radially outer surface of base 1516 angularly aligned with one or more grooves 1550 may be flush with a radially outer surface of anchor 1534 that is between grooves 1550. In various embodiments, an outer diameter of anchor 1534 may be smaller, larger or substantially the same as an outer diameter of base 1516. In some embodiments, the methods and configurations of anchors and lumen interacting devices described herein may facilitate the manufacturing of different types of lumen interacting tools for different types and sizes of lumens.

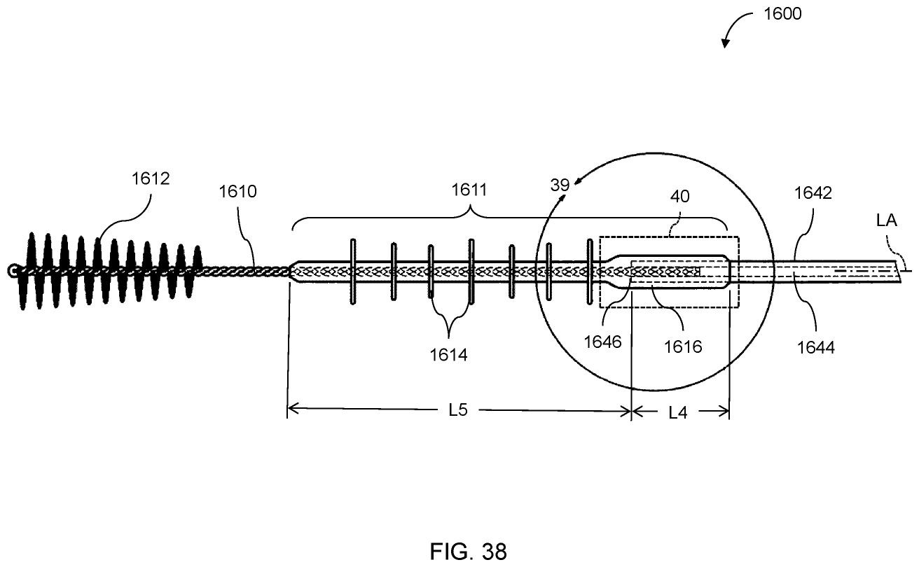

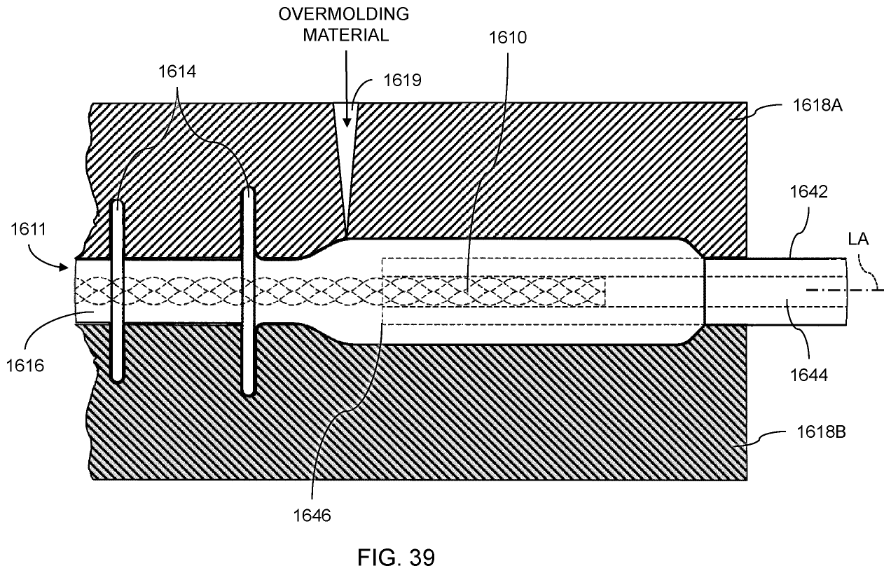

FIG. 38 is a side view of another exemplary lumen cleaning tool 1600 (referred hereinafter as “tool 1600”) for cleaning lumens L (shown in FIG. 11) of medical devices. Tool 1600 may have components previously described in relation to other tools described herein. Tool 1600 may include wiper 1611 and scrubber 1612 that are attached to elongated core 1610 (referred hereinafter as “core 1610”) and disposed at different axial locations along core 1610. Core 1610 may have longitudinal axis LA and may include twisted metallic wires or other type of core 1610. Scrubber 1612 may be a bristle brush. Wiper 1611 may include base 1616 and one or more squeegees 1614 in the form of disc-shaped fins extending radially outwardly from base 1616. Wiper 1611 may be interlocked with core 1610 along longitudinal axis LA of core 1610 using one or more anchors such as tube 1642 and/or other type(s) of anchors. Tube 1642 may be a catheter and may have inner bore 1644. Tube 1642 may be attached to core 1610 and also to wiper 1611 so that longitudinal movement of tool 1600 along longitudinal axis LA may be driven by pushing or pulling on tube 1642 for example.

FIG. 39 is a schematic illustration of part tool 1600 inside of region 39 of FIG. 38 showing wiper 1611 being overmolded onto core 1610 and onto a portion of tube 1642. The portion of core 1610 and the portion of tube 1642 encapsulated by wiper 1611 are shown in broken lines in FIG. 39. Prior to the overmolding process, a portion of core 1610 may be inserted part way into inner bore 1644 of tube 1642. Then, part of tube 1642 and of core 1610 on which wiper 1611 is to be overmolded are inserted into the cavity defined by mold parts 1618A, 1618B. With mold parts 1618A, 1618B (e.g., halves) closed and sealed against core 1610 on one side and against tube 1642 on the other side, the overmolding material in the form of a liquidous resin heated above its glass transition temperature for example may be injected into the cavity defined by mold parts 1618A, 1618B via sprue 1619. The overmolding material may fill the cavity and consequently form wiper 1611 encapsulating a portion of tube 1642 and also a portion of core 1610 disposed outside of tube 1642. For example, base 1616 of wiper 1611 may axially overlap part of tube 1642. In other words, open end 1646 of tube 1642 may be disposed between the opposite axial ends of wiper 1611. After filling the mold cavity and cooling of the overmolding material, mold parts 1618A, 1618B may be opened to release core 1610, wiper 1611 and tube 1642 from mold parts 1618A, 1618B.

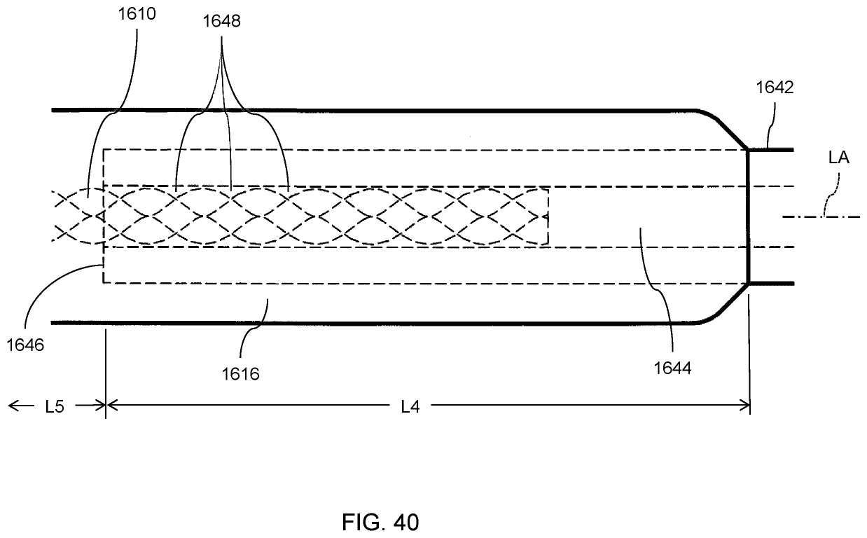

FIG. 40 is an enlarged view of part of tool 1600 in region 40 of FIG. 38. In some embodiments, tube 1642 may be made from PP and wiper 1611 may be made from TPE. In some embodiments, wiper 1611 and tube 1642 may be bonded together by melt adhesion achieved via the overmolding process. For example, overmolding TPE over PP may provide a suitable melt adhesion in some situations. For example, the adhesion of wiper 1611 over tube 1642 over axial span L4 may be superior than the adhesion of wiper 1611 over core 1610 over axial span L5 (shown in FIGS. 38 and 40).

Instead or in addition to the adhesion of wiper 1611 with the exterior surface of tube 1642, the portion of core 1610 received inside of inner bore 1644 may be attached to tube 1642 by any suitable means. In some embodiments, some interstitial space(s) 1648 defined between core 1610 and the surface of inner bore 1644 may get filled with overmolding material during the overmolding process. The presence of overmolding material in interstitial space(s) 1648 may help secure core 1610 to tube 1642 by way of melt adhesion between the overmolding material, the surface of inner bore 1644 and core 1610 for example. Alternatively or in addition, the portion of core 1610 inside of inner bore 1644 may be attached to tube 1642 by way of gluing, heat staking, ultrasonic welding and/or by deformation of the wires of core 1610 for example.

The portion of core 1610 disposed inside of inner bore 1644 may only extend part way along tube 1642. The length of core 1610 inside of inner bore 1644 may be selected based on the attachment desired between core 1610 and inner bore 1644. Accordingly, as shown in FIG. 40, a longitudinal length of tube 1642 along longitudinal axis LA may be greater than a longitudinal length of the portion of core 1610 that is received inside of inner bore 1644 of tube 1642.

In various embodiments, tube 1642 may be attached to wiper 1611 and/or to core 1610 to hinder longitudinal movement of wiper 1611 relative to core 1610. The presence of end 1646 of tube 1642 inside of wiper 1611 may result in tube 1642 defining a protuberance serving as a mechanical interlock obstructing the movement of wiper 1611 along core 1610 especially when tool 1600 is driven along longitudinal axis LA by pushing tube 1642 toward the left direction in FIG. 40.

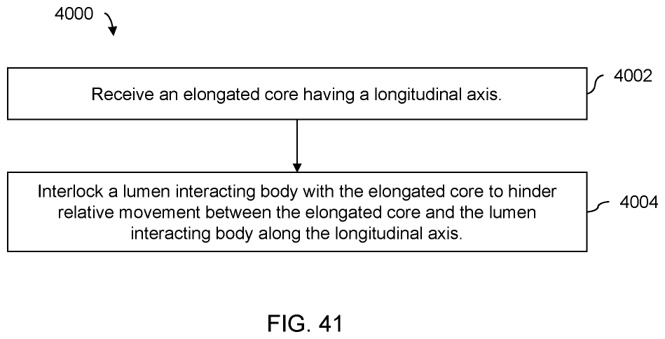

FIG. 41 is a flow diagram of a method 4000 of manufacturing a lumen interacting tool for interacting with (e.g., cleaning, stabilizing) a lumen. Method 4000 may be used to manufacture tools described herein or other lumen interacting tools. Method 4000 may include elements of the tools described herein. Method 4000 may include actions or steps described herein, and/or other actions or steps. In various embodiments, method 4000 may include:
receiving an elongated core having a longitudinal axis (block 4002); and
interlocking a lumen interacting body with the elongated core to hinder relative movement between the elongated core and the lumen interacting body (block 4004).

In some embodiments, interlocking a lumen interacting body (e.g., wiper, lumen stabilizer) with the elongated core may include overmolding the lumen interacting body onto the elongated core using overmolding material. The overmolding material may define one or more beams extending in respective passages formed through the elongated core. Alternatively or in addition, interlocking a lumen interacting body with the elongated core may include at least partially encapsulating a protuberance attached to the elongated core with the lumen interacting body.

In some embodiments, method 4000 may include overmolding the protuberance onto the elongated core.

In some embodiments, the protuberance may include tube 1642 having inner bore 1644, and method 4000 may include inserting core 1610 into inner bore 1644 of tube 1642 before at least partially encapsulating the protuberance with lumen interacting body (e.g., wiper 1611). In some embodiments, at least partially encapsulating the protuberance with the lumen interacting body may include overmolding the lumen interacting body onto the elongated core and onto the protuberance. In some embodiments, overmolding the lumen interacting body onto the elongated core and onto the protuberance may include injecting overmolding material between the elongated core and inner bore 1644 of tube 1642.

The above description is meant to be exemplary only, and one skilled in the relevant arts will recognize that changes may be made to the embodiments described without departing from the scope of the invention disclosed. For example, aspects of the present disclosure may be used with lumen interacting bodies having various configurations and features. The lumen interacting bodies may include one or more outwardly projecting ribs, wings, arms, umbrella-like features, squeegees, fins, disks, O-rings, piston rings, and/or other projections for interacting with lumens L. In some embodiments, the lumen interacting bodies may have a star-like shape when viewed along longitudinal axis LA. The lumen interacting bodies may have different shapes than those shown herein and may be axially symmetrical about longitudinal axis LA, axially non-symmetrical, cylindrical, cone-shaped, frustoconical, oval, spherical, and/or combinations thereof.

The lumen interacting bodies may be made from any suitable material(s) such as TPE, TPR, TPU, TPV, silicone, elastomeric polymer such as ethylene-vinyl acetate (EVA), polyisoprene, urethane, UV curable silicone polymers sold under the trade name KRATON, silicone rubber, fluoropolymers, low density polyethylene (LDPE), and high density polyethylene (HDPE), polyetheretherketone (PEEK) for example. The lumen interacting bodies may, for example, be made from a suitable flexible material, which may have a relatively low-tear resistance. The lumen interacting bodies may be porous or non-porous. In some embodiments, the lumen interacting bodies may have a Shore A hardness of between 20 and 80. In some embodiments, the lumen interacting bodies may have a Shore A hardness greater than 80.

In some embodiments, a lumen interacting body may be made from a relatively soft material that allows it to slide along the elongated core and pulled or pushed over an anchor to engage with (e.g., at least partially encapsulate) the anchor. In some embodiments, the lumen interacting body may be threaded to the anchor. In some embodiments, the lumen interacting body may be formed using an overmolding process, a dipping process, a potting process or 3D printing for example. In some embodiments, the lumen interacting body may be pre-molded into a shape configured to interlock with the anchor(s) and then assembled onto the elongated core as one or more pieces.

Various types and shapes of anchors may be used to interlock the lumen interacting body to the elongated core. The anchors may have different shapes than those shown herein and may be axially symmetrical about longitudinal axis LA, axially non-symmetrical, cylindrical, cone-shaped, frustoconical, oval, spherical, and/or combinations thereof. The anchors may be made from a suitable metallic or polymeric (e.g., PP, PEEK) material. In some embodiments, the material of the anchors may be harder than or of the same hardness as the material of the lumen interacting body. In some embodiments, suitable anchors may be discrete elements separate from the elongated core that are attached to the elongated core using glue, epoxy, welding, crimping, clamping (e.g., between wires of the elongated core), brazing, soldering, friction fit, and threading for example. In some embodiments, suitable anchors may be deposited onto the elongated core using overmolding, 3D printing, brushing on, dipping or dispensing for example. In some embodiments, the anchors may be integrally formed (e.g., have a monolithic construction) with the elongated core. For example, the anchors may include an irregularity such as a loose twist, spiral section, wavy section, flattened section, helical wound or a combination thereof formed in the elongated core.

In some embodiments, the anchors may be integrally formed (e.g., have a monolithic construction) with the lumen interacting body and may be engaged with the elongated core by extending into a passage or other cavity formed in the elongated core. In various embodiments, one or a plurality of axially spaced apart anchors may be used to interlock the lumen interacting body with the elongated core. In situations where a plurality of anchors are used with one lumen interacting body, the anchors may all be of the same shape, size or configuration. Alternatively, anchors of different shapes, sizes or configurations may be used with the same lumen interacting body. In some embodiments, the anchor(s) may be integrally formed (e.g., have a monolithic construction) with the elongated core.

Various types of elongated cores may be used in the tools described herein. In various embodiments, the elongated core may include twisted (intertwined) wires, a single wire, a polymer and/or carbon filament, a tube, rod, a closed wound spring, a multifilar wire twist, stranded wires, and/or a combination thereof.

The present disclosure is intended to cover and embrace all suitable changes in technology. Modifications which fall within the scope of the present invention will be apparent to those skilled in the art, in light of a review of this disclosure, and such modifications are intended to fall within the appended claims. Also, the scope of the claims should not be limited by the preferred embodiments set forth in the examples, but should be given the broadest interpretation consistent with the description as a whole.

1. A lumen cleaning tool for cleaning a lumen of a medical device, the lumen cleaning tool comprising:
an elongated core having a longitudinal axis, the elongated core being defined by wires twisted together;
a wiper attached to the elongated core; and
an anchor interlocking the wiper with the elongated core along the longitudinal axis of the elongated core to hinder relative movement between the elongated core and the wiper along the longitudinal axis.

2. The lumen cleaning tool as defined in claim 1, wherein:
the elongated core includes a tightly twisted section and a loosely twisted section having different axial positions along the elongated core;
the loosely twisted section defines a gap between the wires twisted together;
the wiper is overmolded onto the loosely twisted section of the elongated core;
the anchor includes a beam extending into the gap; and
the beam and the wiper have a monolithic construction.

3. The lumen cleaning tool as defined in claim 2, wherein the gap defines a through passage for the beam to extend transversely through the elongated core.

4. The lumen cleaning tool as defined in claim 1, wherein:
the anchor includes a protuberance attached to the elongated core; and
the wiper at least partially encapsulates the protuberance.

5. (canceled)

6. The lumen cleaning tool as defined in claim 4 or claim 5, wherein the protuberance includes a bristle retained between the wires twisted together.

7-9. (canceled)

10. The lumen cleaning tool as defined in claim 1, wherein:
the anchor includes a helical arrangement of bristles extending radially outwardly from the elongated core; and
the wiper at least partially encapsulates the helical arrangement of bristles.

11. The lumen cleaning tool as defined in claim 1, comprising a plurality of anchors interlocking the wiper with the elongated core along the longitudinal axis of the elongated core, the plurality of anchors being axially spaced apart along the longitudinal axis of the elongated core.

12-19. (canceled)

20. A medical lumen interacting tool for interacting with a lumen, the medical lumen interacting tool comprising:
an elongated core having a longitudinal axis;
a body for interacting with the lumen, the body being attached to the elongated core; and
one or more anchors interlocking the body with the elongated core along the longitudinal axis of the elongated core to hinder relative movement between the elongated core and the body along the longitudinal axis, the one or more anchors including one or both of following:
a beam having a monolithic construction with the body and extending in a passage formed through the elongated core; and
a protuberance attached to the elongated core and being at least partially encapsulated by the body.

21. The medical lumen interacting tool as defined in claim 20, wherein the one or more anchors include the beam.

22. The medical lumen interacting tool as defined in claim 21, wherein:
the elongated core includes wires twisted together; and
the passage is defined by a gap between the wires.

23. The medical lumen interacting tool as defined in claim 22, wherein:
the elongated core includes a tightly twisted section and a loosely twisted section; and
the passage is located in the loosely twisted section.

24-30. (canceled)

31. The medical lumen interacting tool as defined in claim 20, wherein the one or more anchors include the protuberance.

32. (canceled)

33. The medical lumen interacting tool as defined in claim 31, wherein the protuberance includes a bristle.

34. The medical lumen interacting tool as defined in claim 31, wherein the protuberance has a helical shape.

35. The medical lumen interacting tool as defined in claim 34, wherein the protuberance includes a helical arrangement of bristles extending radially outwardly from the elongated core.

36-41. (canceled)

42. The medical lumen interacting tool as defined in claim 31, wherein the elongated core includes a helical coil.

43. The medical lumen interacting tool as defined in claim 42, wherein:
the helical coil includes a first section having a first outer diameter and a second section having a second outer diameter larger than the first outer diameter; and
the protuberance includes the second section.

44-46. (canceled)

47. The medical lumen cleaning tool as defined in claim 20, wherein the body is overmolded onto the elongated core.

48. The medical lumen interacting tool as defined in claim 20, wherein the body is a wiper.

49. The medical lumen interacting tool as defined in claim 20, wherein the body is a lumen stabilizer.

50. A method of manufacturing a medical lumen interacting tool for interacting with a lumen, the method comprising:
receiving an elongated core having a longitudinal axis; and
interlocking a lumen interacting body with the elongated core to hinder relative movement between the elongated core and the lumen interacting body along the longitudinal axis by performing one or both of following:
overmolding the lumen interacting body onto the elongated core using overmolding material, the overmolding material defining a beam extending in a passage formed through the elongated core; and
at least partially encapsulating a protuberance attached to the elongated core with the lumen interacting body.

51. The method as defined in claim 50, wherein interlocking the lumen interacting body with the elongated core includes overmolding the lumen interacting body onto the elongated core using overmolding material, the overmolding material defining the beam extending through the passage formed through the elongated core.

52. The method as defined in claim 51, wherein:
the elongated core includes a pair of wires twisted together; and
the passage is defined by a gap between the pair of wires.

53. The method as defined in claim 52, wherein:
the elongated core includes a tightly twisted section and a loosely twisted section; and
the passage is located in the loosely twisted section.

54. The method as defined in claim 50, wherein interlocking the lumen interacting body with the elongated core includes at least partially encapsulating the protuberance attached to the elongated core with the lumen interacting body.

55. The method as defined in claim 54, wherein at least partially encapsulating the protuberance attached to the elongated core with the lumen interacting body includes ovemolding the lumen interacting body onto the elongated core and onto the protuberance.

56. The method as defined in claim 54, wherein the protuberance includes a bristle.

57. (canceled)

58. The method as defined in claim 50, wherein the elongated core includes a helical coil.

59-61. (canceled)
